(19) United States
(12) Patent Application Publication
(10) Pub. No.: US 2005/0038062 A1
(43) Pub. Date: Feb. 17, 2005

(54) Methods and materials for the treatment of pain comprising opioid antagonists

(76) Inventors: Lindsay H. Burns, San Francisco, CA (US); Grant L. Schoenhard, San Carlos, CA (US)

Correspondence Address: Janet M. McNicholas, Ph.D.;McAndrews, Held & Malloy, Ltd., 34th Floor, 500 West Madison Street, Chicago, IL, 60661, US

(21) Appl. No.: 10/825,257

(22) Filed: Apr. 14, 2004

Related U.S. Application Data
(60) Provisional application No. 60/463,004, filed on Apr. 14, 2003.

(51) Int. Cl. A61K 31/485

(52) U.S. Cl. 514/282

(57) ABSTRACT

Methods and compositions for treating subjects with pain, including neuropathic pain, using opioid antagonists or combinations of opioid antagonists and opioid agonists, including, for example, wherein the amount of an opioid antagonist enhances the neuropathic pain-alleviating potency of an opioid agonist.

### FIELD OF THE INVENTION

The present invention relates to methods and materials for the treatment of pain, including neuropathic pain, using opioid antagonists or combinations of opioid antagonists and opioid agonists, wherein, for example, an antagonist may be administered in an amount that enhances the neuropathic pain-alleviating potency of an agonist, including exogenously administered agonists and/or endogenous agonists. Methods and materials of the invention comprising opioid antagonists or combinations of opioid antagonists and agonists may optionally include one or more additional therapeutic agents, for example, anticonvulsant agents, tricyclic antidepressant agents, anti-dynorphin agents, glutamate receptor antagonist agents, non-steroidal anti-inflammatory drug agents or local anesthetic and/or analgesic agents.

### BACKGROUND OF THE INVENTION

At least two important categories of clinical pain conditions exist: traumatic or inflammatory pain, which results from injury to non-neural tissue, for example, as occurs after surgery or in individuals with arthritis and neuropathic pain, which results from injury to or inflammation of the central or peripheral nervous system. Neuropathic pain, in particular, can be quite severe and not very responsive to analgesics, including narcotic analgesics. For example, neuropathic pain may be a result of an injury to the peripheral nerves, which causes nerve dysfunction. Examples of causes of painful nerve injury include accidental trauma, tumors, cerebral or lumbar spine disease, bum injuries, diabetes, arthritis, post-herpetic inflammation, and surgical procedures, such as endodontic repair. The mechanisms underlying neuropathic pain are poorly understood but a variety of theories have been proposed. For instance, hyperactivity in primary afferent or central nervous system nociceptive neurons, loss of central inhibitory connections, and increased activity in sympathetic efferents have been described as possible mechanisms by which nerve dysfunction can cause neuropathic pain.

Patients afflicted with neuropathic pain experience excruciating, and sometimes debilitating, pain. Although neuropathic pain can be an acute or chronic, this type of pain typically lasts for weeks or even years. Characteristic symptoms of neuropathic pain include hyperesthesia (enhanced sensitivity to a natural stimulus), hyperalgesia (abnormal sensitivity to pain), allodynia (widespread tenderness or hypersensitivity to tactile stimuli), spontaneous burning pain, and/or phantom pain (perception of pain that is non-existent). Hyperesthesia involves an unusual increased or altered sensitivity to sensory stimuli, including for example, acoustic, cerebral, gustatory, muscular, olfactory, onelric, optic or tactile, such as a painful sensation from a normally painless touch stimulus. Allodynia involves an intensified, unpleasant, and painful perception of stimuli triggered by heat or by contact, which is based on a lowering of the pain threshold for these stimuli, including, for example, a non-noxious stimulus to normal skin. Hyperalgesia involves the excessive perception of a variety of stimuli, again based on a lowering of the pain threshold and thus an abnormally increased pain sense, including for example, auditory or muscular. Phantom pain involves a perception of pain in a limb that is non-existent, such as perceived pain in a limb that has been amputated, i.e. phantom limb syndrome.

Neuropathic pain has been associated with a wide range of disease conditions. For instance, long-lasting allodynia has been described as a classical result of the herpes zoster (shingles) infection. Hyperalgesia has been described in AIDS patients at various stages of the disease. Burn wounds have been shown to lead to neuropathic hyperalgesia. Cancer patients receiving cytostatics and vincristine have reported experiencing hyperalgesia as a result of their chemotherapy treatment. A tumor itself can elicit hyperalgesia, perhaps as a result of chronic nerve compression by the tumor. Patients with late stage diabetes have reported hyperalgesia, often experiencing highly painful limbs with simultaneously reduced contact sensitivity of the skin. Allodynia has been reported as the diffuse pain occurring in fibromyaligia. Chronic back pain that results in compression of nerve roots of the spinal cord has also been correlated with neuropathic pain. Migraine pain has been described to include characteristic symptoms exhibited in neuropathic pain.

A variety of proposed mechanisms underlying neuropathic pain have been described, including sensitization of the neurons in the dorsal horn of the spinal cord that receive the initial pain signal. However, the underlying pathology and neuronal mechanisms that cause and propagate neuropathic pain are poorly understood. Commonly used analgesics, such as morphine, codeine, tramadol, and aspirin, have shown limited effectiveness by impacting only some of the symptoms of this type of pain. In addition, the vast majority of patients treated with these analgesics continue to experience pain even when re-administered with the analgesic. Both scientific and clinical experience indicate that advanced states of neuropathic pain are difficult to treat chronically with narcotic analgesics (e.g., morphine). Furthermore, both narcotic and some non-narcotic analgesics (i.e., clonidine, an alpha-2 adrenergic receptor agonist) induce unfavorable side effects, such as constipation (opioid), hypotension (adrenergic), respiratory depression (opioid), pharmacological tolerance (opioid and adrenergic), physical dependence/withdrawal (opioid), sedation (opioid and adrenergic), and dry mouth (adrenergic). These side effect profiles can impact both the physician distribution and patient compliance/acceptance of analgesic therapy.

Because neuropathic pain is non-responsive or only partially responsive to commonly used analgesics, such as morphine, codeine, tramadol, and aspirin, thus, the newer therapeutic options for the treatment of neuropathic pain have been focused on non-narcotic analgesic drugs. One such treatment is capsaicin, the active pain killing constituent of chili peppers. Neuropathic pain syndromes where capsaicin has been shown to be effective include postmastectomy pain [Watson, et al., Pain 35:289-297, (1988); Watson et al., Pain 38: 177-186, (1989)], stump pain [Weintraub, et al., Lancet 336:1003-1004 (1990)], trigeminal neuralgia [Fusco, et al., Anesthesia and Analgesia 74:375-377 (1992)], reflex sympathetic dystrophy [Cheshire, et al., Pain 42:307-311, (1990)], and Guillain-Barre syndrome [Morgenlander, et al., Annals of Neurology 28:199 (1990)]. The major side effect with usage of capsaicin is a burning discomfort upon application and for this reason, patient compliance with capsaicin could be severely impaired.

Tricyclic antidepressants, such as amitriptyline, imipramine, desimipramine, and clomipramine, have been widely reported to have an analgesic effect in neuropathic pain. This analgesic effect is independent of the antidepressant effect and may be dose related. Unfortunately, the analgesic effect of tricyclic antidepressants is tempered by their negative side effect profiles, with somnolence and dry mouth being the predominant side-effects.

Anticonvulsants have historically been reported to produce analgesia in neuropathic pain. In fact, similarities between epilepsy and neuropathic pain were first described by Trousseau in 1885. The first report of analgesia with an anticonvulsant in neuropathic pain was with phenytoin in 1942. Anecdotal evidence points to a similar analgesic effect with lamotrigine, although evidence from randomized controlled trials conducted so far have been mixed. Gabapentin, a structural analogue of the inhibitory neurotransmitter gamma amino butyric acid (GABA), has been demonstrated to reduce neuropathic pain, particularly post herpetic neuralgia and diabetic neuropathy. Carbamazepine, the most frequently used anticonvulsant for neuropathic pain, has been reported to provide analgesia in trigeminal neuralgia and diabetic neuropathy in randomized controlled trials. However, each anticonvulsant differs in their mode of action and as such, multiple attempts at selection and dosing may be required in order to determine the most effective anticonvulsant for treatment. This lengthy process could be a drawback for patient compliance. Furthermore, clinical trials in neuropathic pain using the anticonvulsant, gabapentin, as the sole therapeutic agent have shown only small effects.

Ketamine, a N-methyl D-aspartate (NMDA) receptor antagonist, has recently been reported to impart an analgesic effect in neuropathic pain. However, the use of ketamine is also associated with harmful side-effects that curbs its clinical potential as a viable form of treatment.

To date, there has been limited success in the treatment of neuropathic pain. Despite the variety of compositions proposed or used for the treatment of neuropathic pain, including these listed above, neuropathic pain remains poorly understood and poorly treated. Thus, there remains a significant unmet medical need for effective and sustaining treatments for neuropathic pain.

A variety of patents and publications discuss the treatment of pain which may include neuropathic pain, including a variety of U.S. patents as follows.

Fairbanks et al., U.S. Pat. No. 6,150,419 (the disclosure of which is incorporated herein by reference), discloses the administration of agmantine, an endogenous ligand for alpha 2-adrenergic and imidazoline (I) receptors, for the treatment of neuropathic pain.

Shannon et al., U.S. Pat. No. 5,945,416 (the disclosure of which is incorporated herein by reference), discloses the administration of anticonvulsants, such as olanzapine, carbamazepine, gatapentine, and valproate, for the treatment of neuropathic pain. These agents suffer from limited efficacy and significant side effects (Dray et al, Trends Pharmacol Sci 15(b): 190-197 (1994), the disclosure of which is incorporated herein by reference).

Koppe et al., U.S. Pat. Nos. 3,659,019, 3,954,872, and 4,031,244 (the disclosures of which are incorporated herein by reference), discloses the administration of mexiletine, a sodium channel-blocking agent and antiarrhythmic, for the treatment of neuropathic pain. This agent suffers from limited efficacy and significant side effects (Dray et al, Trends Pharmacol Sci 15(b): 190-197 (1994), the disclosure of which is incorporated herein by reference).

Mayer et al., U.S. Pat. No. 5,502,058 (the disclosure of which is incorporated herein by reference), described a method of producing local analgesia by the administration of local anesthetics, such as bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocaine hydrochloride, procaine hydrochloride, propoxycaine hydrochloride tetracaine, tetracaine hydrochloride, and the like, and/or a nonsteroidal anti-inflammatory drug such as diflusenal, ibuprofen, indomethacin, meclofenamate sodium, mefenamic acid, naproxen, naproxen sodium, oxyphenbutazone, phenylbutazone, piroxicam, sulindac, and tolmetin sodium, for the treatment of neuropathic pain. These agent suffers from limited efficacy and significant side effects (Dray et al, Trends Pharniacol Sci 15(b):190-197 (1994), the disclosure of which is incorporated herein by reference).

Sawynok et al., U.S. Pat. No. 6,211,171 (the disclosure of which is incorporated herein by reference), discloses a method of producing local analgesia by administration of tricyclic antidepressants for the treatment of neuropathic pain.

Kreek et al., U.S. Pat. No. 4,769,372 (the disclosure of which is incorporated herein by reference), discloses the administration of a combination of an opioid analgesic or antitussive, such as morphine, meperidine, oxycodone, hydromorphone, codeine and hydrocodone, together with an opioid antagonist, such as naloxone, naloxone glucuronide and nalmefene glucuronide, for treatment of chronic pain or chronic cough in a patient.

Mayer et al., U.S. Pat. No. 5,352,683 (the disclosure of which is incorporated herein by reference), discloses the administration of N-methyl-D-aspartate (NMDA) receptor antagonists, such as dextromethorphan, dextrorphan, and ketamine, for the treatment of neuropathic pain.

Pert et al., U.S. Pat. No. 5,534,495 (the disclosure of which is incorporated herein by reference), describes a treatment for non-HIV neuropathic pain by administering an effective amount of a peptide that is capable of blocking the loss, destruction, or dysfunction of the cellular constituents that lead to non-HIV neuropathic pain.

Justice et al., U.S. Pat. No. 5,587,454 (the disclosure of which is incorporated herein by reference), discloses the use of omega C-onopeptides to produce analgesia for certain types of neuropathic pain.

Caruso et al., U.S. Pat. No. 6,187,338 (the disclosure of which is incorporated herein by reference), discloses the administration of tramadol prior to the administration of a N-methyl-D-aspartate (NMDA) receptor blocker, for the enhanced treatment of neuropathic pain.

Fairbanks et al., U.S. Pat. No. 6,054,461 (the disclosure of which is incorporated herein by reference), discloses the administration of moxonidine, an antihypertensive, for the treatment of neuropathic pain.

Caruso et al., U.S. Pat. No. 6,406,716 (the disclosure of which is incorporated herein by reference), discloses the administration of a combination of an anticonvulsant, such as gabapentin, together with a N-methyl-D-aspartate (NMDA) receptor antagonist, for the treatment of neuropathic pain.

Levine et al., U.S. Pat. No. 6,525,062 (the disclosure of which is incorporated herein by reference), discloses the administration of a combination of nalbuphine together with an opioid antagonist, such as naloxone, naltrexone, and nalmefene, for the treatment of pain, including neuropathic pain.

Benedyk et al., U.S. Pat. No. 6,489,350 (the disclosure of which is incorporated herein by reference), discloses the administration of heteroarylmethanesulfonamides, such as zonisamide (a T- and L- type calcium channel blocker), for the treatment of neuropathic pain.

Rundfeldt et al., U.S. Pat. No. 6,117,900 (the disclosure of which is incorporated herein by reference), discloses the administration of retigabine, an anticonvulsant, for the treatment of neuropathic pain.

Crain and Shen in U.S. Pat. Nos. 5,472,943; 5,512,578 reissued as RE 36,457; 5,580,876; 5,767,125; 6,096,756; and 6,362,194 (the disclosures of which are incorporated herein by reference) describe methods and compositions of opioids for selectively enhancing the analgesic potency of a bimodally-acting opioid agonist and simultaneously attenuating anti-analgesia, hyperalgesia, hyperexcitability, physical dependence and/or tolerance effects associated with the administration of the bimodally-acting opioid agonist, by administering to a subject an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist. Also disclosed are methods and compositions of opioids for treating pain in a subject by administering to the subject an analgesic or sub-analgesic amount of a bimodally-acting opioid agonist and an amount of an excitatory opioid receptor antagonist effective to enhance the analgesic potency of the bimodally-acting opioid agonist and simultaneously attenuate anti-analgesia, hyperalgesia, hyperexcitability, physical dependence and/or tolerance effects of the bimodally-acting opioid agonist.

### SUMMARY OF THE INVENTION

The present invention provides compositions and methods for the treatment of pain, including neuropathic pain, comprising opioid antagonists. Such antagonists are used alone or in combination with opioid agonists and/or other therapeutic agents for the treatment of pain, particularly neuropathic pain.

In addition to compositions comprising an opioid antagonist or combination of opioid antagonist and opioid agonist, compositions of the invention optionally comprise one or more additional therapeutic agents, for example, an anticonvulsant or anti-epileptic, a glutamate receptor (or subunit thereof) antagonist, a tricyclic antidepressant agent, capsaicin, a local anesthetic and/or analgesic, an anti-dynorphin agent, a nicotinic receptor agonist, or a non-narcotic analgesic, such as a non-steroidal anti-inflammatory drug.

In one aspect, the present invention is directed to compositions that comprise an opioid antagonist or combination of an opioid antagonist and an opioid agonist, including wherein the amount of antagonist is effective to enhance the neuropathic pain-alleviating potency of the agonist. Such compositions optionally additionally comprise one or more pharmaceutically acceptable carriers or excipients.

In another aspect, the present invention is also directed to methods for treating pain in a subject, including a subject with neuropathic pain, by administering to the subject an opioid antagonist or combination of an opioid agonist and an opioid antagonist, including wherein the antagonist is administered in an amount effective to enhance the neuropathic pain-alleviating potency of the agonist. Such methods of the invention optionally comprise the additional administration of one or more additional therapeutic agents, for example, an anticonvulsant or anti-epileptic, a glutamate receptor (or subunit thereof) antagonist, a tricyclic antidepressant agent, capsaicin, a local anesthetic and/or analgesic, an anti-dynorphin agent or a non-narcotic analgesic, such as a non-steroidal anti-inflammatory drug.

In another aspect, the present invention is directed to methods for enhancing the potency of opioid agonists by administering the agonists along with opioid antagonists to subjects with neuropathic pain in amounts of antagonist that are effective to enhance the neuropathic pain-alleviating potency of the administered agonist.

In another aspect, the present invention is directed to compositions for administration to subjects with neuropathic pain wherein the compositions comprise an analgesic or subanalgesic amount of an opioid agonist and an amount of an opioid antagonist effective to enhance the neuropathic pain-alleviating potency of the agonist.

In another aspect, the present invention is also directed to compositions for administration to neuropathic pain patients that comprise in one or more compositions an opioid antagonist alone or in combination with an opioid agonist and/or a local anesthetic, the antagonist is administered in an amount effective to enhance the neuropathic pain-alleviating potency of an endogenous opioid agonist or an administered (e.g., exogenous) opioid agonist. Such compositions additionally comprise one or more additional therapeutic agents as listed above and optionally a pharmaceutically acceptable carrier or excipient.

In another aspect, the present invention is directed to methods for treating neuropathic pain in patients in need thereof by administering to the patient a composition comprising an amount of an opioid antagonist effective to enhance the neuropathic pain-alleviating potency of an opioid agonist, whereby the neuropathic pain is alleviated.

Pain, including neuropathic pain, is alleviated (e.g., ameliorated, attenuated, reduced or diminished), by compositions and methods of the invention, for example, as measured by an alleviation (e.g., amelioration, attenuation, reduction, or diminishment) of one or more of hyperesthesia, hyperalgesia, allodynia, spontaneous burning pain or phantom pain. In one aspect, one or more of such symptoms are not from the administration of a therapeutic agent (e.g., opioid agonist).

### DETAILED DESCRIPTION OF THE INVENTION

The present invention provides methods for treating pain, including neuropathic pain. The present invention provides pain-alleviating compositions, including neuropathic pain-alleviating compositions, that comprise an opioid antagonist (e.g., excitatory opioid receptor antagonist). Such compositions additionally preferentially comprise an opioid agonist (e.g., a bimodally-acting opioid agonist), and optionally a pharmaceutically acceptable carrier or excipient for administration to a subject, preferably a human, in need thereof. The present invention also provides methods for treating a subject in pain, including neuropathic pain, comprising administering (a) an opioid agonist, (b) an opioid antagonist, including in an amount effective to enhance the neuropathic pain-alleviating potency of the agonist, and optionally (c) a pharmaceutically acceptable carrier or excipient for administration to the subject, preferably a human, in need thereof, whereby the pain, including neuropathic pain, is alleviated.

The present invention is directed to novel neuropathic pain-alleviating compositions, dosage forms, and kits with an excitatory opioid receptor antagonist in combination with a biomodally-acting opioid agonist, wherein the antagonist is present in an amount effective to enhance the neuropathic pain-alleviating potency of the agonist. The invention further relates to methods for administering to human subjects such neuropathic pain-alleviating compositions, dosage forms, and kits.

The present invention also provides methods for treating a subject with pain, including neuropathic pain, comprising administering (a) a local anesthetic or analgesic, (b) an amount of opioid antagonist effective to enhance the pain-alleviating potency of an endogenous opioid agonist, and optionally (c) a pharmaceutically acceptable carrier or excipient for administration to the subject, preferably a human, in need thereof, whereby the pain is alleviated. Such methods optionally include additionally administering an opioid agonist, and in such methods, the amount of antagonist is effective to enhance the pain-alleviating potency of the administered agonist.

Preferred indications contemplated for employing the compositions and methods for treating neuropathic pain presented herein include any indication, condition, disorder or disease that involves neuropathic pain, including migraine, neuropathic pain of diabetes, diabetic neuropathy (e.g., diabetic peripheral neuropathy), shingles, burn injuries, ophthalmic injuries, oral nerve injury, reflex sympathetic dystrophy (RSD), post-herpetic neuralgia, arthritis, or neuropathic pain by injury, amputation or overuse, including sciatica and low back pain. The compositions and methods for treating pain, including neuropathic pain, presented herein alleviate (e.g., ameliorate, attenuate, reduce or diminish) at least one symptom of pain, particularly neuropathic pain, such as, for example, allodynia, hyperesthesia, hyperalgesia, spontaneous burning pain, or phantom pain syndrome. Such symptoms may be characteristic of, associated with, or arising out of the pain syndrome. Symptoms from nerve injury or damage, including sensory nerve injury or damage, are pain, including pain that persists after signs of original injury or damage disappear (e.g., neuropathic pain), numbness, tingling, burning and/or loss (e.g., dullness) of feeling. Compositions and methods of the present invention are useful for the alleviation (e.g., amelioration, attenuation, reduction or diminishment) of such symptoms. In preferred embodiments, such symptoms are not from the administration of any therapeutic agent (e.g., opioid agonist).

Compositions according to the invention that are neuropathic pain-alleviating compositions are those that when administered result in an alleviation (e.g., amelioration, attenuation, reduction or diminishment) of at least one symptom of existing neuropathic pain and/or the suppression or inhibition of neuropathic pain which would otherwise ensue from an imminent neuropathic pain-causing event. Such neuropathic pain-alleviating compositions include neuropathic pain-suppressing and neuropathic pain-inhibiting compositions.

An effective pain-alleviating amount refers to an amount of opioid antagonist or combination of opioid agonist and antagonist which elicits alleviation (e.g., amelioration, attenuation, reduction or diminishment) of at least one symptom of pain upon administration to a subject (e.g., patient) in need thereof.

An effective neuropathic pain-alleviating amount refers to an amount of opioid antagonist or combination of opioid agonist and antagonist which elicits alleviation (e.g., amelioration, attenuation, reduction or diminishment) of at least one symptom of neuropathic pain upon administration to a subject (e.g., patient) in need thereof.

An amount of opioid antagonist that enhances the pain-alleviating potency, such as the neuropathic pain-alleviating potency, of opioid agonist is the amount that when added to an analgesic or subanalgesic amount of agonist results upon administration in a greater alleviation (e.g., amelioration, attenuation, reduction or diminishment) of at least one symptom of pain, such as neuropathic pain, than the alleviation of that symptom resulting from administration of that agonist alone (i.e., without that amount of antagonist).

An amount of opioid antagonist that enhances the pain-alleviating potency of an endogenous opioid agonist is the amount that when administered alone or with another therapeutic agent, such as local anesthetic and/or opioid agonist, results in a greater alleviation (e.g., amelioration, attenuation, reduction or diminishment) of at least one symptom of pain than the alleviation of that symptom without that amount of antagonist.

Opioids refer to compounds or compositions, including metabolites of the compounds or compositions, that bind to specific opioid receptors and have agonist (activation) or antagonist (inactivation) effects at the opioid receptors.

Inhibitory opioid receptors refer to opioid receptors that mediate inhibitory opioid receptor functions, such as analgesia.

Excitatory opioid receptors refer to opioid receptors that mediate excitatory opioid receptor functions, such as anti-analgesic effects, physical dependence, tolerance, hyperexcitability and hyperalgesia.

Opioid receptor agonist or opioid agonist refers to an opioid compound or composition, including any active metabolite of such compound or composition, that binds to and activates opioid receptors on neurons that mediate pain.

A bimodally-acting opioid agonist refers to an opioid agonist that binds to and activates both inhibitory and excitatory opioid receptors on neurons that mediate pain.

An opioid receptor antagonist or opioid antagonist refers to an opioid compound or composition, including any active metabolite of such compound or composition, that binds to and blocks opioid receptors on neurons that mediate pain. An opioid antagonist attenuates (e.g., blocks, inhibits, prevents, or competes with) the action of an opioid agonist.

An excitatory opioid receptor antagonist refers to an opioid which binds to and acts as an antagonist to excitatory but not inhibitory opioid receptors on neurons that mediate pain.

An agonist refers to a compound or composition capable of combining with (e.g., binding to) receptors to initiate pharmacological actions.

Pharmaceutically acceptable refers to those compounds, materials, compositions, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

Pharmaceutically acceptable salts refer to derivatives of the disclosed compounds wherein the compounds are modified by making at least one acid or base salt thereof, and includes inorganic and organic salts.

An analgesic amount refers of opioid agonist to an amount of the opioid agonist which causes analgesia in a patient administered the opioid receptor agonist alone, and includes standard doses of the agonist which are typically administered to cause analgesia (e.g. mg doses).

A subanalgesic amount of opioid agonist refers to an amount which does not cause analgesia in a patient administered the opioid receptor agonist alone, but when used in combination with a potentiating or enhancing amount of opioid antagonist, results in analgesia.

An effective antagonistic amount of opioid antagonist refers to an amount that effectively attenuates (e.g. blocks, inhibits, prevents, or competes with) the analgesic activity of an opioid agonist.

A therapeutically effective amount of a neuropathic pain-alleviating composition refers to an amount that elicits alleviation of at least one symptom of neuropathic pain upon administration to a patient in need thereof.

A therapeutically effective amount of a neuropathic pain-alleviating composition refers to an amount that elicits alleviation of at least one symptom of neuropathic pain upon administration to a patient in need thereof.

Potency may refer to the strength of a drug or drug treatment in producing desired effects, for example, analgesia and/or the alleviation of, for example, hyperalgesia, allodynia, hyperesthesia or phantom pain. Potency also may refer to the effectiveness or efficacy of a drug treatment in eliciting desired effects, for example, analgesia and/or alleviation of hyperalgesia, allodynia, hyperesthesia, spontaneous burning pain or phantom pain. For example, enhanced potency may refer to the lowering of a dose in achieving desired effects or to an increased therapeutic benefit including that not previously seen, for example, where the increased therapeutic benefit is eliciting desired effects such as analgesia and/or alleviation of hyperalgesia (e.g., hyperalgesia not associated with or resulting from administration of an opioid agonist, such as chronic administration of the opioid agonist), allodynia, hyperesthesia, spontaneous burning pain or phantom pain from oral administration, oral formulation or oral dosage form. In therapeutics, for example, potency may refer to the relative pharmacological activity of a compound or a composition.

An anticonvulsant or anti-epileptic refers to a pharmaceutically acceptable agent or therapeutic agent that treats or prevents or arrests seizures, such as in epilepsy. Anticonvulsants include cabamazepine, phenytoin, valproate, ethosuximide, gabapentin, lamotrigine, levetiracetam, tiagabine, toprimate, and zonisamide.

A local anesthetic and/or analgesic refers to a pharmaceutically acceptably agent or therapeutic agent that is administered locally to a nerve and anesthetizes local nerves, thereby conferring pain relief. Local anesthetics include bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocaine hydrochloride, procaine hydrochloride propoxycaine hydrochloride tetracaine or tetracaine hydrochloride and their 1,2 and 1,3 hydroxy derivatives; meperidine, diphenoxylate, loperimide, fentanyl, sufentanil, alfentanil, remifentanil, and the like.

A glutamate receptor (or subunit thereof) antagonist refers to a pharmaceutically acceptable agent or therapeutic agent that is an antagonist of an NMDA, AMPA, kainate or metabotropic receptor or subtype or subunit of such receptor. Glutamate receptor antagonists include ketamine, MK801, memantine, dextromethorphan, dextrorphan, LY293558, LY382884, amantadine, agmatine, aptiganel, gavestinel, selfotel, 7-chlorokynurenate, MRZ 2/579, MDL 105,519, riluzole, CPP, AP5, APV, NBQX, CNQX or trans-ACPD.

A tricyclic antidepressant agent refers to a chemical group of antidepressant drugs that share a 3-ringed nucleus; e.g., amitriptyline, imipramine, desipramine or nortriptyline that confers relief of pain.

An anti-dynorphin agent refers to a drug or biologic that removes, degrades or interferes with dynorphin and includes anti-dynorphin antibodies, soluble kappa opioid receptors, or soluble kappa opioid receptor fusion proteins.

A nicotinic receptor agonist refers to a pharmaceutically acceptable agent or a therapeutic agent that is an agonist of a neuronal nicotinic receptor (NNR) capable of binding to peripheral NNRs, including those which do not readily cross the blood-brain barrier such as those described in U.S. Patent Application Publications 2004/0010018 and 2003/0216448.

An NSAID refers to a non-steriodal anti-inflammatory drug and includes anti-inflammatory drugs such as aspirin, members of the cycloxgenease I, II and III inhibitors, and includes naproxen sodium, diclofenac and misoprostol, valdecoxib, diclofenac, celecoxib, sulindac, oxaprozin, diflunisal, piroxicam, indomethacin, meloxicam, ibuprofen, naproxen, mefenamic acid, nabumetone, ketorolac, choline or magnesium salicylates, rofecoxib, tolmetin sodium, phenylbutazone, oxyphenbutzone, meclofenamate sodium or diflusenal.

Hyperesthemia refers to a symptom wherein a patient with neuropathic pain experiences enhanced sensitivity to natural stimulus. Hyperesthemia refers to a symptom or condition involving increased or altered (e.g., abnormal or pathological) sensitivity to a sensory stimulus, for example, as of the skin to touch or ear to sound.

Allodynia refers to a symptom wherein a patient with neuropathic pain experiences widespread tenderness or hypersensitivity to tactile stimuli. Allodynia refers to a symptom or condition in which ordinarily nonpainful stimuli evoke pain, for example, a painful sensation in response to a normally innocuous stimulus.

Hyperalgesia refers to a symptom wherein a patient with neuropathic pain experiences abnormal and heightened sensitivity to pain. Hyperalgesia refers to a symptom or condition involving a strong or heightened painful sensation to a mildly or lowered painful stimulus. Hyperalgesia may refer to a hyperalgesia from injury or damage, including nerve injury or damage or hyperalgesia resulting from or associated with administration of a therapeutic agent, including drugs, wherein the agent is not an opioid agonist.

Spontaneous pain, including spontaneous burning pain, refers to a symptom wherein a patient with neuropathic pain experiences pain without external stimuli. Spontaneous pain, including spontaneous burning pain, refers to a symptom or condition involving a painful sensation in the absence of an external stimulus.

Phantom pain syndrome refers to a symptom wherein a patient with neuropathic pain experiences a perception of pain that is non-existent, such as in phantom limb syndrome.

Excitatory opioid receptor antagonists in the present neuropathic pain-alleviating compositions are opioids which bind to and act as antagonists to excitatory but not inhibitory opioid receptors on nociceptive neurons that mediate pain. That is, excitatory opioid receptor antagonists are compounds which bind to excitatory opioid receptors and selectively block excitatory opioid receptor functions of nociceptive types of DRG neurons at 1,000 to 10,000-fold lower concentrations than are required to block inhibitory opioid receptor functions in these or similar neurons. Excitatory opioid receptor antagonists may also be identified by measuring their effect on the action potential duration (APD) of dorsal root ganglion (DRG) neurons in tissue cultures. In this regard, excitatory opioid receptor antagonists are compounds that selectively block prolongation of the APD of DRG or similar neurons (i.e., excitatory effects) but not the shortening of the APD of DRG neurons (i.e., inhibitory effects) elicited by a bimodally-acting opioid receptor agonist.

The antagonist in the present neuropathic pain-alleviating compositions may be present in its original form or in the form of a pharmaceutically acceptable salt. The antagonists in the present neuropathic pain-alleviating compositions include: naltrexone, naloxone, nalmefene, methylnaltrexone, methiodide, nalorphine, naloxonazine, nalide, nalmexone, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, levorphanol, meptazinol, dezocine, or their pharmacologically effective esters or salts. Preferred opioid antagonists include naltrexone, nalmefene, naloxone, or mixtures thereof. Particularly preferred is nalmefene or naltrexone.

In general, for neuropathic pain-alleviating compositions, dosage forms, kits and methods according to the invention, an opioid antagonist is provided in an amount from about 1 fg to about 1.0 mg or from about 1 fg to about 1 μg. Alternatively, the opioid antagonist is provided in an amount from at least about 0.000001 mg to about or less than about 0.5 or 1.0 mg, 0.00001 mg to about or less than about 0.5 or 1.0 mg, 0.0001 mg to about or less than about 0.5 or 1.0 mg, or at least about 0.001 mg to about or less than about 0.5 or 1.0 mg, or at least about 0.01 mg to about or less than about 0.5 or 1.0 mg, or at least about 0.1 mg to about or less than about 0.5 or 1.0 mg. Preferred ranges of opioid antagonists also include: from about 0.000001 mg to less than 0.2 mg; from about 0.00001 mg to less than 0.2 mg; from about 0.0001 mg to less than 0.2 mg; from about 0.001 mg to less than 0.2 mg; from about 0.01 mg to less than 0.2 mg; or from about 0.1 mg to less than 0.2 mg. Additional preferred ranges of opioid antagonists include: from about 0.0001 mg to about 0.1 mg; from about 0.001 mg to about 0.1 mg; from about 0.01 mg. to about 0.1 mg; from about 0.001 mg to about 0.1 mg; from about 0.001 mg to about 0.01 mg; or from about 0.01 mg to about 0.1 mg.

In a preferred dosage form, the maximum amount of antagonist is 1 mg, alternatively less than 1 mg, alternatively 0.99 mg, alternatively 0.98 mg, alternatively 0.97 mg, alternatively 0.96 mg, alternatively 0.95 mg, alternatively 0.94 mg, alternatively 0.93 mg, alternatively 0.92 mg, alternatively 0.91 mg, alternatively 0.90 mg, alternatively 0.89 mg, alternatively 0.88 mg, alternatively 0.87 mg, alternatively 0.86 mg, alternatively 0.85 mg, alternatively 0.84 mg, alternatively 0.83 mg, alternatively 0.82 mg, alternatively 0.81 mg, alternatively 0.80 mg, alternatively 0.79 mg, alternatively 0.78 mg, alternatively 0.77 mg, alternatively 0.76 mg, alternatively 0.75 mg, alternatively 0.74 mg, alternatively 0.73 mg, alternatively 0.72 mg, alternatively 0.71 mg, alternatively 0.70 mg, alternatively 0.69 mg, alternatively 0.68 mg, alternatively 0.67 mg, alternatively 0.66 mg, alternatively 0.65 mg, alternatively 0.64 mg, alternatively 0.63 mg, alternatively 0.62 mg, alternatively 0.61 mg, alternatively 0.60 mg, alternatively 0.59 mg, alternatively 0.58 mg, alternatively 0.57 mg, alternatively 0.56 mg, alternatively 0.55 mg, alternatively 0.54 mg, alternatively 0.53 mg, alternatively 0.52 mg, alternatively 0.51 mg, alternatively 0.50 mg.

Additionally, the maximum amount of antagonist in the preferred dosage form is less than 0.5 mg, alternatively 0.49 mg, alternatively 0.48 mg, alternatively 0.47 mg, alternatively 0.46 mg, alternatively 0.45 mg, alternatively 0.44 mg, alternatively 0.43 mg, alternatively 0.42 mg, alternatively 0.41 mg, alternatively 0.40 mg, alternatively 0.39 mg, alternatively 0.38 mg, alternatively 0.37 mg, alternatively 0.36 mg, alternatively 0.35 mg, alternatively 0.34 mg, alternatively 0.33 mg, alternatively 0.32 mg, alternatively 0.31 mg, alternatively 0.30 mg, alternatively 0.29 mg, alternatively 0.28 mg, alternatively 0.27 mg, alternatively 0.26 mg, alternatively 0.25 mg, alternatively 0.24 mg, alternatively 0.23 mg, alternatively 0.22 mg, alternatively 0.21 mg, alternatively 0.20 mg, alternatively 0.19 mg, alternatively 0.18 mg, alternatively 0.17 mg, alternatively 0.16 mg, alternatively 0.15 mg, alternatively 0.14 mg, alternatively 0.13 mg, alternatively 0.12 mg, alternatively 0.11 mg, alternatively 0.10 mg, alternatively 0.09 mg, alternatively 0.08 mg, alternatively 0.07 mg, alternatively 0.06 mg, alternatively 0.05 mg, alternatively 0.04 mg, alternatively 0.03 mg, alternatively 0.02 mg, alternatively 0.01 mg, alternatively 0.009 mg, alternatively 0.008 mg, alternatively 0.007 mg, alternatively 0.006 mg, alternatively 0.005 mg, alternatively 0.004 mg, alternatively 0.003 mg, alternatively 0.002 mg, alternatively 0.001 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg, alternatively 0.0001 mg.

Additionally, the minimum amount of antagonist in the preferred dosage form is 0.0001 mg, alternatively 0.0002 mg, alternatively 0.0003 mg, alternatively 0.0004 mg, alternatively 0.0005 mg, 0.0006 mg, alternatively 0.0007 mg, alternatively 0.0008 mg, alternatively 0.0009 mg, alternatively 0.001 mg, alternatively 0.002 mg, alternatively 0.003 mg, alternatively 0.004 mg, alternatively 0.005 mg, alternatively 0.006 mg, alternatively 0.007 mg, alternatively 0.008 mg, alternatively 0.009 mg, alternatively 0.01 mg, alternatively 0.011 mg, alternatively 0.012 mg, alternatively 0.013 mg, alternatively 0.014 mg, alternatively 0.015 mg, alternatively 0.016 mg, alternatively 0.017 mg, alternatively 0.018 mg, alternatively 0.019 mg, alternatively 0.02 mg, alternatively 0.021 mg, alternatively 0.022 mg, alternatively 0.023 mg, alternatively 0.024 mg, alternatively 0.025 mg, alternatively 0.026 mg, alternatively 0.027 mg, alternatively 0.028 mg, alternatively 0.029 mg, alternatively 0.03 mg, alternatively 0.031 mg, alternatively 0.032 mg, alternatively 0.033 mg, alternatively 0.034 mg, alternatively 0.035 mg, alternatively 0.036 mg, alternatively 0.037 mg, alternatively 0.038 mg, alternatively 0.039 mg, alternatively 0.04 mg, alternatively 0.041 mg, alternatively 0.042 mg, alternatively 0.043 mg, alternatively 0.044 mg, alternatively 0.045 mg, alternatively 0.046 mg, alternatively 0.047 mg, alternatively 0.048 mg, alternatively 0.049 mg, alternatively 0.05 mg, alternatively 0.051 mg, alternatively 0.052 mg, alternatively 0.053 mg, alternatively 0.054 mg, alternatively 0.055 mg, alternatively 0.056 mg, alternatively 0.057 mg, alternatively 0.058 mg, alternatively 0.059 mg, alternatively 0.06 mg, alternatively 0.061 mg, alternatively 0.062 mg, alternatively 0.063 mg, alternatively 0.064 mg, alternatively 0.065 mg, alternatively 0.066 mg, alternatively 0.067 mg, alternatively 0.068 mg, alternatively 0.069 mg, alternatively 0.07 mg, alternatively 0.071 mg, alternatively 0.072 mg, alternatively 0.073 mg, alternatively 0.074 mg, alternatively 0.075 mg, alternatively 0.076 mg, alternatively 0.077 mg, alternatively 0.078 mg, alternatively 0.079 mg, alternatively 0.08 mg, alternatively 0.081 mg, alternatively 0.082 mg, alternatively 0.083 mg, alternatively 0.084 mg, alternatively 0.085 mg, alternatively 0.086 mg, alternatively 0.087 mg, alternatively 0.088 mg, alternatively 0.089 mg, alternatively 0.09 mg, alternatively 0.091 mg, alternatively 0.092 mg, alternatively 0.093 mg, alternatively 0.094 mg, alternatively 0.095 mg, alternatively 0.096 mg, alternatively 0.097 mg, alternatively 0.098 mg, alternatively 0.099 mg, alternatively 0.1 mg, alternatively 0.11 mg, alternatively 0.12 mg, alternatively 0.13 mg, alternatively 0.14 mg, 0.15 mg, alternatively 0.16 mg, alternatively 0.17 mg, alternatively 0.18 mg, alternatively 0.19 mg, alternatively 0.2 mg, alternatively 0.21 mg, alternatively 0.22 mg, alternatively 0.23 mg, alternatively 0.24 mg, alternatively 0.25 mg, alternatively 0.26 mg, alternatively 0.27 mg, alternatively 0.28 mg, alternatively 0.29 mg, alternatively 0.3 mg, alternatively 0.31 mg, alternatively 0.32 mg, alternatively 0.33 mg, alternatively 0.34 mg, alternatively 0.35 mg, alternatively 0.36 mg, alternatively 0.37 mg, alternatively 0.38 mg, alternatively 0.39 mg alternatively 0.40 mg, alternatively 0.41 mg, alternatively 0.42 mg, alternatively 0.43 mg, alternatively 0.44 mg, alternatively 0.45 mg, alternatively 0.46 mg, alternatively 0.47 mg, alternatively 0.48 mg, alternatively 0.49 mg, alternatively 0.5 mg, alternatively 0.51 mg, alternatively 0.52 mg, alternatively 0.53 mg, alternatively 0.54 mg, alternatively 0.55 mg, alternatively 0.56 mg, alternatively 0.57 mg, alternatively 0.58 mg, alternatively 0.59 mg, alternatively 0.6 mg, alternatively 0.61 mg, alternatively 0.62 mg, alternatively 0.63 mg, alternatively 0.64 mg, alternatively 0.65 mg, alternatively 0.66 mg, alternatively 0.67 mg, alternatively 0.68 mg, alternatively 0.69 mg, alternatively 0.7 mg, alternatively 0.71 mg, alternatively 0.72 mg, alternatively 0.73 mg, alternatively 0.74 mg, alternatively 0.75 mg, alternatively 0.76 mg, alternatively 0.77 mg, alternatively 0.78 mg, alternatively 0.79 mg, alternatively 0.8 mg, alternatively 0.81 mg, alternatively 0.82 mg, alternatively 0.83 mg, alternatively 0.84 mg, alternatively 0.85 mg, alternatively 0.86 mg, alternatively 0.87 mg, alternatively 0.88 mg, alternatively 0.89 mg, alternatively 0.9 mg, alternatively 0.91 mg, alternatively 0.92 mg, alternatively 0.93 mg, alternatively 0.94 mg, alternatively 0.95 mg, alternatively 0.96 mg, alternatively 0.97 mg, alternatively 0.98 mg, alternatively 0.99 mg.

In a more preferred dosage form, the maximum amount of antagonist is less than 0.20 mg, alternatively 0.19 mg, alternatively 0.18 mg, alternatively 0.17 mg, alternatively 0.16 mg, alternatively 0.15 mg, alternatively 0.14 mg, alternatively 0.13 mg, alternatively 0.12 mg, alternatively 0.11 mg, alternatively 0.10 mg, alternatively 0.09 mg, alternatively 0.08 mg, alternatively 0.07 mg, alternatively 0.06 mg, alternatively 0.05 mg, alternatively 0.04 mg, alternatively 0.03 mg, alternatively 0.02 mg, alternatively 0.01 mg, alternatively 0.009 mg, alternatively 0.008 mg, alternatively 0.007 mg, alternatively 0.006 mg, alternatively 0.005 mg, alternatively 0.004 mg, alternatively 0.003 mg, alternatively 0.002 mg, alternatively 0.001 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg.

Additionally, the minimum amount of antagonist in the preferred dosage form is 0.0001 mg, alternatively 0.0002 mg, alternatively 0.0003 mg, alternatively 0.0004 mg, alternatively 0.0005 mg, 0.0006 mg, alternatively 0.0007 mg, alternatively 0.0008 mg, alternatively 0.0009 mg, alternatively 0.001 mg, alternatively 0.002 mg, alternatively 0.003 mg, alternatively 0.004 mg, alternatively 0.005 mg, alternatively 0.006 mg, alternatively 0.007 mg, alternatively 0.008 mg, alternatively 0.009 mg, alternatively 0.01 mg, alternatively 0.011 mg, alternatively 0.012 mg, alternatively 0.013 mg, alternatively 0.014 mg, alternatively 0.015 mg, alternatively 0.016 mg, alternatively 0.017 mg, alternatively 0.018 mg, alternatively 0.019 mg, alternatively 0.02 mg, alternatively 0.021 mg, alternatively 0.022 mg, alternatively 0.023 mg, alternatively 0.024 mg, alternatively 0.025 mg, alternatively 0.026 mg, alternatively 0.027 mg, alternatively 0.028 mg, alternatively 0.029 mg, alternatively 0.03 mg, alternatively 0.031 mg, alternatively 0.032 mg, alternatively 0.033 mg, alternatively 0.034 mg, alternatively 0.035 mg, alternatively 0.036 mg, alternatively 0.037 mg, alternatively 0.038 mg, alternatively 0.039 mg, alternatively 0.04 mg, alternatively 0.041 mg, alternatively 0.042 mg, alternatively 0.043 mg, alternatively 0.044 mg, alternatively 0.045 mg, alternatively 0.046 mg, alternatively 0.047 mg, alternatively 0.048 mg, alternatively 0.049 mg, alternatively 0.05 mg, alternatively 0.051 mg, alternatively 0.052 mg, alternatively 0.053 mg, alternatively 0.054 mg, alternatively 0.055 mg, alternatively 0.056 mg, alternatively 0.057 mg, alternatively 0.058 mg, alternatively 0.059 mg, alternatively 0.06 mg, alternatively 0.061 mg, alternatively 0.062 mg, alternatively 0.063 mg, alternatively 0.064 mg, alternatively 0.065 mg, alternatively 0.066 mg, alternatively 0.067 mg, alternatively 0.068 mg, alternatively 0.069 mg, alternatively 0.07 mg, alternatively 0.071 mg, alternatively 0.072 mg, alternatively 0.073 mg, alternatively 0.074 mg, alternatively 0.075 mg, alternatively 0.076 mg, alternatively 0.077 mg, alternatively 0.078 mg, alternatively 0.079 mg, alternatively 0.08 mg, alternatively 0.081 mg, alternatively 0.082 mg, alternatively 0.083 mg, alternatively 0.084 mg, alternatively 0.085 mg, alternatively 0.086 mg, alternatively 0.087 mg, alternatively 0.088 mg, alternatively 0.089 mg, alternatively 0.09 mg, alternatively 0.091 mg, alternatively 0.092 mg, alternatively 0.093 mg, alternatively 0.094 mg, alternatively 0.095 mg, alternatively 0.096 mg, alternatively 0.097 mg, alternatively 0.098 mg, alternatively 0.099 mg, alternatively 0.1 mg, alternatively 0.11 mg, alternatively 0.12 mg, alternatively 0.13 mg, alternatively 0.14 mg, 0.15 mg, alternatively 0.16 mg, alternatively 0.17 mg, alternatively 0.18 mg, alternatively 0.19 mg, alternatively less than 0.2 mg.

Any minimum amount and any maximum amount of antagonist in the dosage form, including, for example, as specified above, may be combined to define a range of amounts, providing that the minimum selected is equal to or less than the maximum selected.

The amount of an opioid antagonist in the present neuropathic pain-alleviating compositions effective to enhance the neuropathic pain-alleviating potency of an opioid agonist can be less than an effective antagonistic amount. The effective amount of an opioid antagonist in the present neuropathic pain-alleviating compositions can be about 1.0 mg. The effective neuropathic pain-alleviating amount of an opioid antagonist in the present neuropathic pain-alleviating compositions can be less than 1.0 mg. The effective amount of an opioid antagonist in the present neuropathic pain-alleviating compositions can be about 0.1 mg. The effective neuropathic pain-alleviating amount of an opioid antagonist in the present neuropathic pain-alleviating compositions can be less than 0.1 mg. The effective neuropathic pain-alleviating amount of an opioid antagonist in the present neuropathic pain-alleviating compositions can be about 0.01 mg. The effective neuropathic pain-alleviating amount of an opioid antagonist in the present neuropathic pain-alleviating compositions can be less than 0.01 mg. The effective neuropathic pain-alleviating amount of an opioid antagonist in the present neuropathic pain-alleviating compositions can be more than 0.01 mg. The effective neuropathic pain-alleviating amount of an opioid antagonist in the present neuropathic pain-alleviating compositions can be about 0.001 mg. The effective neuropathic pain-alleviating amount of an opioid antagonist in the present neuropathic pain-alleviating compositions can be less than 0.001 mg. The effective neuropathic pain-alleviating amount of an opioid antagonist in the present neuropathic pain-alleviating compositions can be more than 0.001 mg, including, for example, 0.002 mg, 0.003 mg, 0.004 mg, 0.005 mg, 0.006 mg, 0.007 mg, 0.008 mg, 0.009 mg or 0.010 mg. The effective neuropathic pain-alleviating amount of an opioid antagonist in the present neuropathic pain-alleviating compositions can be about 0.0001 mg. The effective neuropathic pain-alleviating amount of an opioid antagonist in the present neuropathic pain-alleviating compositions can be more than 0.0001 mg. The effective amount of an opioid antagonist in the present neuropathic pain-alleviating compositions can be about 0.00001 mg. The effective neuropathic pain-alleviating amount of an opioid antagonist in the present neuropathic pain-alleviating compositions can be less than 0.00001 mg. The effective neuropathic pain-alleviating amount of an opioid antagonist in the present neuropathic pain-alleviating compositions can be more than 0.00001 mg. The effective amount of an opioid antagonist in the present neuropathic pain-alleviating compositions can be about 0.000001 mg. The effective neuropathic pain-alleviating amount of an opioid antagonist in the present neuropathic pain-alleviating compositions can be less than 0.000001 mg. The effective neuropathic pain-alleviating amount of an opioid antagonist in the present neuropathic pain-alleviating compositions can be more than 0.000001 mg.

Opioid agonists, including bimodally-acting opioid agonists, are optionally but preferably present in pain-alleviating compositions, including neuropathic pain-alleviating compositions, of the invention. Bimodally-acting opioid agonists in the present neuropathic pain-alleviating compositions are opioid agonists that bind to and activate both inhibitory and excitatory opioid receptors on nociceptive neurons which mediate pain. Activation of inhibitory receptors by the agonists causes analgesia. Activation of excitatory receptors by the agonists results in for example, hyperexcitability and/or hyperalgesia, as well as development of physical dependence, tolerance or other undesirable side effects. Bimodally-acting opioid agonists may be identified by measuring the opioid's effect on the action potential duration (APD) of dorsal root ganglion (DRG) neurons in tissue cultures. In this regard, bimodally-acting opioid agonists are compounds which elicit prolongation of the APD of DRG or similar neurons at pM-nM concentrations (i.e. excitatory effects), and shortening of the APD of DRG or similar neurons at μM concentrations (i.e., inhibitory effects).

The agonist in the present neuropathic pain-alleviating compositions may be present in its original form or in the form of a pharmaceutically acceptable salt. The agonists in the present neuropathic pain-alleviating compositions include: alfentanil, allylprodine, alphaprodine, anileridine, apomorphine, apocodeine, benzylmorphine, bezitramide, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, narceine, nicomorphine, norlevorphanol, normethadone, nalorphine, normorphine, norpipanone, ohmefentanyl, opium, oxycodone, oxymorphone, papaveretum, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, salts thereof, mixtures of any of the foregoing, mixed mu-agonists/antagonists, mu-antagonist combinations, or others known to those skilled in the art. Preferred agonists for human use are morphine, hydrocodone, oxycodone, codeine, fentanyl (and its relatives), hydromorphone, meperidine, methadone, oxymorphone, propoxyphene or tramadol, or mixtures thereof. Particularly preferred contemplated agonists are morphine, hydrocodone, oxycodone or tramadol. Opioid agonists include exogenous or endogenous opioids. Endogenous opioid agonists include endorphin, beta-endorphin, enkephalin, met-enkephalin, dynorphin, orphanin FQ, neuropeptide FF, nociceptin, endomorphin, endormorphin-1, endormorphin-2. Preferred opioid agonists for oral administration, oral formulations and/or oral dosage forms include oxycodone, oxymorphone, hydromorphone or hydrocodone, or mixtures thereof, wherein oxycodone is particularly preferred.

The agonist may be present in an amount that is analgesic or subanalgesic (e.g., non-analgesic) in the human subject. The agonist is administered in dosage forms containing from about 0.1 to about 300 mg of agonist. The agonist, in conjunction with antagonist, is included in the dosage form in an amount sufficient to produce the desired effect upon the process or condition of pain, including neuropathic pain, such as alleviation (e.g., amelioration, attenuation, reduction or diminishment) of at least one symptom of pain, including neuropathic pain. Symptoms include, for example, hyperesthesia, allodynia, spontaneous burning pain, phantom pain, or hyperalgesia.

Preferred combinations of an opioid antagonist and opioid agonist in the present neuropathic pain-alleviating compositions are naltrexone and morphine; naltrexone and oxycodone; naltrexone and hydrocodone; naltrexone and tramadol; nalmefene and morphine; nalmefene and oxycodone; nalmefene and tramadol; nalmefene and hydrocodone; naloxone and morphine; naloxone and oxycodone; naloxone and hydrocodone; naloxone and tramadol; respectively.

In an embodiment of the invention, the amount of antagonist in the dosage form is less than an effective amount to antagonize an exogenous or endogenous agonist, but such an amount is effective to enhance the pain-enhancing potency, including the neuropathic pain-enhancing potency, of the agonist and optionally but preferably is effective to attenuate an adverse effect of the agonist, for example, tolerance, withdrawal, dependence and/or addiction. In another aspect of the invention, the method further comprises administering the opioid agonist, in either a combined dosage form with the antagonist or in a separate dosage form. Still another aspect of the invention provides an immediate release solid oral dosage form comprising one or more pharmaceutical excipients, a dose of an opioid agonist and a low dose of an opioid antagonist, wherein the opioid agonist and opioid antagonist are release concurrently when placed in an aqueous environment. The opioid antagonist and opioid agonist can be formulated as immediate release, (IR), controlled release (CR) and/or sustained released (SR) formulations. Formulations can have components that are combinations of IR and/or CR and/or SR components.

The combination dosage forms of the present neuropathic pain-alleviating compositions can be formulated to provide a concurrent release of the opioid antagonist in combination with opioid agonist and/or other therapeutic agent generally throughout at least a majority of the delivery profile for the formulation. As used herein, the terms “concurrent release” and “released concurrently” mean that the agonist and antagonist are released in in vitro dissolution assays in an overlapping manner. The respective beginnings of release of each agent can but need not necessarily be simultaneous. Concurrent release will occur when the majority of the release of the first agent overlap a majority of release of the second agent. A desired portion of each active pharmaceutical ingredient may be released within a desired time. The desired portions may be, for example, 5%, 50% or 90%, or some other percentage between 1% and The desired time may be in minutes or hours, for example, 10 minutes, 20 minutes, 30 minutes, or 45 minutes, or some other time. The desired portion and the desired time may be varied by the inclusion of formulants for the controlled release or sustained release of any therapeutic agent(s).

The optimum amounts of the opioid antagonist administered in combination with an opioid agonist or other therapeutic agent will of course depend upon the particular antagonist and agonist or other agent used, the excipient chosen, the route of administration, and/or the pharmacokinetic properties of the patient being treated. Effective administration levels of antagonist and agonist or other agent will vary upon the state and circumstances of the patient being treated. As those skilled in the art will recognize, many factors that modify the action of an active ingredient will be taken into account by a treating physician, such as the age, body weight, sex, diet, and condition of the patient, the lapse of time between the condition or injury and the administration of the present neuropathic pain-alleviating compositions, and the administration technique. A person of ordinary skill in the art will be able to ascertain the optimal dosage for a given set of conditions in view of the disclosure herein.

The opioid agonist and/or antagonist can be present in the present neuropathic pain-alleviating compositions as an acid, base, pharmaceutically acceptable salt, or a combination thereof. The pharmaceutically acceptable salt embraces inorganic or organic salts. Examples of pharmaceutically acceptable salts include, but are not limited to, mineral or organic acid salts. The pharmaceutically acceptable salts include the conventional non-toxic salts made, for example, from non-toxic inorganic or organic acids. For example, such conventional non-toxic salts include those derived from inorganic acids such as hydrochloric, hydrobromic, sulfuric, sulfonic, sulfamic, phosphoric, nitric and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucuronic, and other acids. Other pharmaceutically acceptable salts and variants include mucates, phosphate (dibasic), phosphate (monobasic), acetate trihydrate, bi(heptafluorobutyrate), bi(methylcarbamate), bi(pentafluoropropionate), mesylate, bi(pyridine-3-carboxylate), bi(trifluoroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed.(Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are all hereby incorporated by reference. Additional representative salts include hydrobromide, hydrochloride, mucate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate, bi(pyridine-3-carboxylate), bi(trifluoroacetate), bitartrate, chlorhydrate, fumarate, and sulfate pentahydrate.

In an embodiment, the present pain-alleviating compositions, including neuropathic pain-alleviating compositions, further comprise at least one anticonvulsant or anti-epileptic agent. Any therapeutically effective anticonvulsant may be used according to the invention. For extensive listings of anticonvulsants, see, e.g., Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083 (the disclosures of which are incorporated herein by reference). Representative anticonvulsants that can be used herein include lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan and L-5-hydroxytryptophan. Currently marketed anticonvulant/anti-epileptic drugs include Keppra®, Lamictol®, Neurontin®, Tegretol®, Carbatrol®, Topiramate®, Trileptal®, and Zonegran®.

With regard to dosage levels, the anticonvulsant is present at a neuropathic pain-alleviating amount or an amount that is not pain-alleviating alone but is pain-alleviating in combination with an opioid agonist and opioid antagonist according to the invention. This amount is at a level corresponding to the generally recommended adult human dosages for a particular anticonvulsant. The effective neuropathic pain-alleviating amount of the opioid antagonist and the opioid agonist can be present at a level that potentiates the neuropathic pain-alleviating effectiveness of the anticonvulsant. Specific dosage levels for the anticonvulsants that can be used herein as given, inter alia, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosure of all are incorporated herein by reference. As is well known to one of ordinary skill in the art, there can be a wide variation in the dosage level of the anticonvulsant, wherein the dosage level depends to a large extent on the specific anticonvulsant being administered. These amounts can be determined for a particular drug combination, in accordance with this invention, by employing routine experimental testing.

In an embodiment, the present pain-alleviating compositions, including neuropathic pain-alleviating compositions, further comprise at least one non-narcotic analgesic, such as a nonsteroidal anti-inflammatory agent (NSAID). Representative nonsteroidal anti-inflammatory agents include aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxican, sulindac, tolmetin, and zomepirac. Currently marketed NSAIDs include Celebrex®, Vioxx®, Anaprox®, Arthrotec®, Bextra®, Cataflam®, Celebrex®, Clinoril®, DayPro®, Dolobid®, Feldene®, Indocin®, Mobic®, Motrin®, Negprelen®, Naprosyn®, Ponstel®, Relafen®, Toradolg®.

With regard to dosage levels, the non-narcotic analgesic is present in a neuropathic pain-alleviating amount or an amount that is not pain-alleviating alone but is pain-alleviating in combination with an opioid agonist and opioid antagonist according to the invention. This amount is at a level corresponding to the generally recommended adult human dosages for a particular non-narcotic analgesic. The effective neuropathic pain-alleviating amount of the opioid antagonist and the opioid agonist can be present at a level that potentiates the neuropathic pain-alleviating effectiveness of the non-narcotic analgesic. Specific dosage levels for the non-narcotic analgesic that can be used herein as given, inter alia, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of all are incorporated herein by reference. As is well known to one of ordinary skill in the art, there can be a wide variation in the dosage level of the non-narcotic analgesic, wherein the dosage level depends to a large extent on the specific non-narcotic analgesic being administered. These amounts can be determined for a particular drug combination in accordance with this invention by employing routine experimental testing.

The pain-alleviating compounds or compositions, including neuropathic pain-alleviating compounds or compositions, presented herein may be compounded, for example, with the usual non-toxic, pharmaceutically acceptable excipients, carriers, diluents or other adjuvants. The choice of adjuvants will depend upon the active ingredients employed, the physical form of the composition, the route of administration, and other factors. Routes of administration may include oral, intravenous, intrathecal or topical, preferably oral.

The excipients, binders, carriers, and diluents which can be used include water, glucose, lactose, natural sugars such as sucrose, glucose, or corn sweeteners, sorbitol, natural and synthetic gums such as gum acacia, tragacanth, sodium alginate, and gum arabic, gelatin, mannitol, starches such as starch paste, corn starch, or potato starch, magnesium trisilicate, talc, keratin, colloidal silica, urea, stearic acid, magnesium stearate, dibasic calcium phosphate, crystalline cellulose, methyl cellulose, carboxymethyl cellulose, polyethylene glycol, waxes, glycerin, and saline solution, among others.

Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The dosage forms can also comprise one or more acidifying agents, adsorbents, alkalizing agents, antiadherents, antioxidants, binders, buffering agents, colorants, complexing agents, diluents or fillers, direct compression excipients, disintegrants, flavorants, fragrances, glidants, lubricants, opaquants, plasticizers, polishing agents, preservatives, sweetening agents, or other ingredients known for use in pharmaceutical preparations.

Acidifying agents are a compound used to provide an acidic medium for product stability. Such compounds include, by way of example and without limitation, acetic acid, amino acid, citric acid, fumaric acid and other alpha hydroxy acids, hydrochloric acid, ascorbic acid, nitric acid, phosphoric acid, and others known to those skilled in the art.

Adsorbents are agents capable of holding other molecules onto their surface by physical or chemical (chemisorption) means. Such compounds include, by way of example and without limitation, powdered and activated charcoal, zeolites, and other materials known to one of ordinary skill in the art.

Alkalizing agent are compounds used to provide an alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those skilled in the art.

Antiadherent are agents that prevents the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

Antioxidants are agents which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

Binders are substances used to cause adhesion of powder particles in solid dosage formulations. Such compounds include, by way of example and without limitation, acacia, alginic acid, carboxymethylcellulose sodium, poly(vinylpyrrolidone), compressible sugar (e.g., NuTab), ethylcellulose, gelatin, liquid glucose, methylcellulose, povidone and pregelatinized starch and other materials known to one of ordinary skill in the art.

When needed, binders may also be included in the dosage forms. Exemplary binders include acacia, tragacanth, gelatin, starch, cellulose materials such as methyl cellulose, HPMC, HPC, HEC and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, gelatin, cellulosics in nonaqueous solvents, combinations thereof and others known to those skilled in the art. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

Buffering agents are compounds used to resist changes in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

Sweetening agents are compounds used to impart sweetness to a preparation. Such compounds include, by way of example and without limitation, aspartame, dextrose, glycerin, mannitol, saccharin sodium, sorbitol, sucrose, and other materials known to one of ordinary skill in the art.

Diluents or fillers are inert substances used to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, and starch and other materials known to one of ordinary skill in the art.

Direct compression excipients are compounds used in compressed solid dosage forms. Such compounds include, by way of example and without limitation, dibasic calcium phosphate (e.g., Ditab) and other materials known to one of ordinary skill in the art.

Disintegrants are compounds used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays such as bentonite, microcrystalline cellulose (e.g., Avicel), methyl cellulose, carboxymethylcellulose calcium, sodium carboxymethylcellulose, alginic acid, sodium alginate, cellulose polyacrilin potassium (e.g., Amberlite), alginates, sodium starch glycolate, gums, agar, guar, locust bean, karaya, xanthan, pectin, tragacanth, agar, bentonite, and other materials known to one of ordinary skill in the art.

Glidants are agents used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art.

Lubricants are substances used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art.

Opaquants are compounds used to render a coating opaque. An opaquant may be used alone or in combination with a colorant. Such compounds include, by way of example and without limitation, titanium dioxide, talc and other materials known to one of ordinary skill in the art.

Polishing agents are compounds used to impart an attractive sheen to solid dosage forms. Such compounds include, by way of example and without limitation, camauba wax, white wax and other materials known to one of ordinary skill in the art.

Colorants are compounds used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, ferric oxide, other FD&C dyes and natural coloring agents such as grape skin extract, beet red powder, beta-carotene, annato, carmine, turmeric, paprika, and other materials known to one of ordinary skill in the art. The amount of coloring agent used will vary as desired.

Flavorants are compounds used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those skilled in the art. Particularly contemplated flavors are the grape and cherry flavors and citrus flavors such as orange.

Complexing agents include for example EDTA disodium or its other salts and other agents known to one of ordinary skill in the art.

Exemplary fragrances include those generally accepted as FD&C grade.

Exemplary preservatives include materials that inhibit bacterial growth, such as Nipagin, Nipasol, alcohol, antimicrobial agents, benzoic acid, sodium benzoate, benzyl alcohol, sorbic acid, parabens, isopropyl alcohol and others known to one of ordinary skill in the art.

Solid dosage forms of the invention can also employ one or more surface active agents or cosolvents that improve wetting or disintegration of the core and/or layer of the solid dosage form.

Plasticizers can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. The PEG based plasticizers are available commercially or can be made by a variety of methods, such as disclosed in Poly(ethylene glycol)Chemistry Biotechnical and Biomedical Applications (J. M. Harris, Ed.; Plenum Press, NY) the disclosure of which is hereby incorporated by reference.

Solid dosage forms of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for the solid pharmaceutical compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl □-aminopropionates and 2-alkylimidazoline quaternary ammonium salts; and others known to one of ordinary skill in the art; and mixtures thereof.

A water soluble coat or layer can be formed to surround a solid dosage form or a portion thereof. The water soluble coat or layer can either be inert or drug-containing. Such a coat or layer will generally comprise an inert and non-toxic material which is at least partially, and optionally substantially completely, soluble or erodible in an environment of use. Selection of suitable materials will depend upon the desired behavior of the dosage form. A rapidly dissolving coat or layer will be soluble in the buccal cavity and/or upper GI tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the disclosures of which are hereby incorporated by reference. In some embodiments, the rapidly dissolving coat or layer will be soluble in saliva, gastric juices, or acidic fluids.

Materials which are suitable for making the water soluble coat or layer include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those skilled in the art. Other lamina-forming materials that can be used for this purpose include poly(vinyl alcohol), poly(ethylene oxide), gelatin, glucose and saccharides. The water soluble coating can comprise other pharmaceutical excipients that may or may not alter the way in which the water soluble coating behaves. The artisan of ordinary skill will recognize that the above-noted materials include film-forming polymers.

A water soluble coat or layer can also comprise hydroxypropyl methylcellulose, which is supplied by Dow under its Methocel E-15 trademark. The materials can be prepared in solutions having different concentrations of polymer according to the desired solution viscosity. For example, a 2% WN aqueous solution of Methocel™ E-15 has a viscosity of about 13-18 cps at 20° C.

For transcutaneous or transdermal administration, the compounds may be combined with skin penetration enhancers such as propylene glycol, polyethylene glycol, isopropanol, ethanol, oleic acid, N-methylpyrrolidone, or others known to those skilled in the art, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, or others known to those skilled in the art, to provide the composition in gel form, which can be dissolved in solvent such as methylene chloride, evaporated to the desired viscosity, and then applied to backing material to provide a patch.

For intravenous, intramuscular, subcutaneous, intrathecal, epidural, perineural or intradermal administration, the active ingredients may be combined with a sterile aqueous solution. The solution may be isotonic with the blood of the recipient. Such formulations may be prepared by dissolving one or more solid active ingredients in water containing physiologically compatible substances such as sodium chloride, glycine, or others known to those skilled in the art, and/or having a buffered pH compatible with physiological conditions to produce an aqueous solution, and/or rendering the solution sterile. The formulations may be present in unit dose containers such as sealed ampoules or vials.

For topical (e.g., dermal or subdermal) or depot administration, the active ingredients may be formulated with oils such as cottonseed, hydrogenated castor oil and mineral oil; short chain alcohols as chlorobutanol and benzyl alcohol; also including polyethylene glycols, polysorbates; polymers such as sucrose acetate isobutyrate, caboxymethocellusose and acrylates; buffers such as dihydrogen phosphate; salts such as sodium chloride and calcium phosphate; and other ingredients included but not exclusive to povidone, lactose monohydrate, magnesium stearate, myristyo-gamma-picolinium; and water.

A solid dosage form of the invention can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

Various other components, in some cases not otherwise listed above, can be added to the present formulation for optimization of a desired active agent release profile including, by way of example and without limitation, glycerylmonostearate, nylon, cellulose acetate butyrate, d,1-poly(lactic acid), 1,6-hexanediamine, diethylenetriamine, starches, derivatized starches, acetylated monoglycerides, gelatin coacervates, poly (styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

The present, pain-alleviating compositions, including neuropathic pain-alleviating compositions, can be formulated in capsules, tablets, caplets, or pills. Such capsules, tablets, caplets, or pills of the present neuropathic pain-alleviating compositions can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Similarly, the carrier or diluent may include any sustained release material known in the art, such as glyceryl monostearate or glyceryl distearate, alone or mixed with a wax. The formulations of the invention may be formulated so as to provide quick, sustained, or delayed release of the active ingredient after administration to the patient by employing procedures well known in the art.

Controlled release or sustained-release dosage forms, as well as immediate release dosage forms are specifically contemplated. Controlled release or sustained release as well as immediate release compositions in liquid forms in which a therapeutic agent may be incorporated for administration orally or by injection are also contemplated.

Pain-alleviating compositions, including neuropathic pain-alleviating compositions, presented herein can be administered from about one time daily to about six times daily, two times daily to about four times daily, or one time daily to about two times daily.

Pain-alleviating compositions, including neuropathic pain-alleviating compositions, presented herein preferably comprise at least one colloidal dispersion system, additive or preservative, diluent, binder, plasticizer, or slow release agent.

It should be understood that compounds used in the art of pharmaceutical formulation generally serve a variety of functions or purposes. Thus, whether a compound named herein is mentioned only once or is used to define more than one term herein, its purpose or function should not be construed as being limited solely to the named purpose(s) or function(s).

The present pain-alleviating compounds or compositions, including neuropathic pain-alleviating compounds or compositions, may be in admixture with an organic or inorganic carrier or excipient suitable for administration in enteral or parenteral applications, such as orally, topically, transdermally, by inhalation spray, rectally, by subcutaneous, intravenous, intramuscular, subcutaneous, intrathecal, epidural, perineural, intradermal, intraocular injection or infusion techniques. Preferably, such compositions are in the form of a topical, intravenous, intrathecal, epidural, perineural, or oral formulation. More preferably, such compositions are in the form of an intrathecal, epidural or perineural formulation. Even more preferably, such compositions are in the form of an intravenous formulation. Most preferably, such compositions are in the form of an oral formulation.

Pain, including neuropathic pain, may result from a number of separate etiologies. Generally, progression of such pain may be treated according to any of the methods described herein. However, in many cases it will be preferable to treat the pain in a manner that addresses its specific source. For example, when the pain is traceable to injury of a particular nerve fiber, it may be appropriate to treat such pain either by perineural application of compound to the affected nerve or by dermal application of compound to the affected region. Presented herein are non-limiting, representative indications for which treatment according to the methods of the present invention may have particular therapeutic utility. The indications described below are by no means exhaustive, but are presented to illustrate some of the various therapeutic situations in which the neuropathic pain-alleviating compositions presented herein can be used.

(I) Ophthalmic indications. The eye is a heavily innervated organ. The cornea in particular is heavily innervated with C-fibers, containing an estimated 3-4000 fiber endings per mm² compared to an estimated 3-600 fiber endings per mm² of skin. Injury of the nerve fibers can lead to neuropathic pain of ophthalmic origin. In accordance with the present invention, the eye may be treated, preferably topically, with the neuropathic pain-alleviating compositions presented herein to prevent progression of neuropathy. Application of the neuropathic pain-alleviating compositions presented herein may be achieved by topical administration to the eye, or, in more severe cases, by means of suprachoroidal administration. Chronic implanted therapeutics are also indicated after ophthalmic surgery, such as after surgery for detached retina or macular holes, where nerve damage may result.

(II) Dental indications. Delivery of the neuropathic pain-alleviating compositions presented herein to regions of dental repair, such as endodontic repair concomitant to a root canal procedure, may be desirable as a means of preventing progression of dental neuropathy. Here, the neuropathic pain-alleviating compositions presented herein may be included in or added to one or more of the polymer based materials or resins inserted into the root canal after removing the pulp from the region, in accordance with standard techniques known in the art.

(III) Burn injury. Burn injuries are characterized by primary hyperalgesia to thermal and mechanical stimuli. In accordance with the principles discussed above, treatment of burned regions with the neuropathic pain-alleviating compositions presented herein may reduce progression of the hyperalgesic response by interfering with signal transduction mechanisms of nociceptor sensory receptors. In this embodiment of the invention, the neuropathic pain-alleviating compositions can be applied directly to the affected regions, or can be applied in a formulation that includes a protective biopolymer matrix, such as an artificial skin matrix.

(IV) Reflex sympathetic dystrophy (RSD). RSD is thought to be due to abnormalities in the peripheral nervous system, and more particularly, to sensitization of cutaneous somatosensory afferents. Sympathetic outflow is thought to activate foci of ectopic neural hyperexcitability. Treatment of this condition to prevent its progression may be effected by any of the various dermal (topical) or subdermal routes of delivery discussed above. Perineural delivery may also be indicated for this condition.

(V) Post-herpetic neuralgia. Post-herpetic neuralgia is characterized, in its acute phase, by intraneural inflammation which can cause damage to primary afferent axons. This damage may result in abnormal sensitivity to cutaneous stimuli. In general, the mode of treatment to prevent progression of abnormal sensitivity will depend on the location in the body of the affected nerve(s). Perineural or topical delivery of therapeutic N-type calcium channel blocking compound is indicated in this condition.

(VI) Diabetic neuropathy. Neuropathy of primary afferent axons in long nerves is found in diabetic patients. This results in the dying-back and attempted regeneration of distal tips of primary afferent axons of these nerves. Nociceptor sensitization may ensue. Such sensitization and its progression may be treated according to one or more of the treatment methods described herein. In particular, perineural or topical application of the neuropathic pain-alleviating compositions presented herein will be indicated, in accord with the location of the affected nerve and nerve beds.

(VII) Arthritis. Arthritis is characterized by enhanced sensation of pain via articular afferents. The neuropathic pain-alleviating compositions presented herein find utility in treatment of such pain according to the principles set forth in the present invention. Generally, in treating articular afferents, the neuropathic pain-alleviating compositions presented herein will be administered perineurally, including topically, in the vicinity of the affected joint.

(VIII) Cancer Chemotherapy. Peripheral neuropathy, a tingling, numbness or pain in the extremities, can be one effect of chemotherapy. Vincristine, Cisplatin, Taxol, and Vinblastine are chemotherapeutics that are more likely to cause this type of neuropathic pain when given long-term in high doses. The neuropathic pain-alleviating compositions presented herein find utility in treatment of such pain according to the principles set forth in the present invention.

The following examples are provided for illustrative purposes and are not to be construed to limit the scope of the claims in any manner whatsoever.

### EXAMPLE 1

### In vivo Studies of Neuropathic Pain

This example describes an evaluation of neuropathic pain in an animal model. An in vivo rat L5/L6 spinal nerve ligation (SNL) study is conducted as described by Kim and Chung (1992). These in vivo experiments to evaluate neuropathic pain with compounds and compositions according to the invention as prescribed herein are conducted on male Sprague-Dawley rats (Harlan; Indianapolis, Ind.) that are 200-225 g at the time of the L5/L6 surgery as described below. The rats are maintained in a climate-controlled room on a 12-h light/dark cycle (lights on at 06:00 h); food and water are provided ad libiturn. The testing is performed in accordance with accepted policies and guidelines for the handling and use of laboratory animals.

A. SNL Study

In an initial study, the effects of intrathecally delivered morphine and naloxone or morphine alone are tested after ligation of the L5 and L6 spinal nerves in rats. At one week, after a neuropathic pain syndrome of thermal hyperalgesia and tactile allodynia is stabilized, rats receive daily intrathecal injections of vehicle, morphine alone, morphine plus naloxone or naloxone alone for seven days. This study tests the ability of a combination of agonist plus antagonist, for example, morphine plus naloxone (a) to alleviate thermal hyperalgesia more potently than agonist (e.g., morphine) alone, (b) to produce an anti-allodynia effect (i.e., attenuate, alleviate or reduce allodynia) not seen with agonist (e.g., morphine) alone, and (c) maintain analgesia over a seven day study period in which agonist (e.g., morphine) administration alone results in tolerance. The animals first undergo intrathecal implantation of a catheter that allows for drug delivery at the lumbar (L5) spinal cord region. Animal baseline withdrawals from thermal and tactile stimuli are recorded. Animals then undergo the L5/L6 ligation surgery as described below and are tested for tactile and thermal hypersensitivities, for example, on day 3 through day 7 after the surgery, and then, for example, on day 8, animals begin the twice daily intrathecal administration of vehicle or drug substances in doses as described below. Animals are tested, for example, from about 20 to about 30 minutes after the first daily intrathecal dose for hyperalgesic and anti-allodynia effects for a total of 7 days as described below.

For intrathecal injection of drug substance, for example, opioid agonists and/or antagonists, rats are prepared using an amended method described by Yaksh and Rudy (Yaksh and Rudy, 1976), and generally illustrated as follows. While under halothane anesthesia, an 8 cm length of 32 gauge tubing is inserted in the Sprague-Dawley rats to the level of the lumbar enlargement via an incision made in the atlanto-occipital membrane. The catheter is then secured to the musculature at the site of incision, which is then closed. The rats receive 4.4 mg/kg of gentamycin (intramuscular administration) as a prophylactic precaution and are allowed 5-7 days for recovery before experimentation begins, for example, before L5/L6 ligation surgery begins as described below. Any rats exhibiting signs of motor deficiency are euthanized. Intrathecally administered substances are dissolved in saline (to a total volume of 5 μl) prior to administration through a length of tubing connecting the intrathecal catheter with the injection syringe. Progress of the injection is determined by observing the movement of an air bubble through a calibrated length of the tubing. The catheter is cleared by flushing with 9 μl saline.

For these experiments, morphine, when given either as the sole drug or in combination with naltrexone to the rats, is administered to the rats at a dosage ranging from 1-30 micrograms, if given intrathecally. Naltrexone, when given either as the sole drug or in combination with morphine to the rats, is administered to the rats at a dosage ranging from 1 femtogram (fg) to 30 nanograms (ng) if given intrathecally. For other experiments, if given systemically, morphine, when given either as the sole drug or in combination with naltrexone to the rats, is administered to the rats at a dosage ranging from 1 mg/kg to 20 mg/kg. If given systemically, naltrexone, when given either as the sole drug or in combination with morphine to the rats, is administered to the rats at a dosage ranging from 1 femtogram to 300 nanograms. Administration of drug or vehicle to the rat is performed, for example, both on a BID or QID basis. For these experiments, morphine sulfate is purchased from Sigmia (St. Louis, Mo.) and naltrexone hydrochloride is purchased from Tocris (Ellisville, Mo.). These substances are dissolved in sterile 0.9% physiological saline from Baxter (Deerfield, Ill.).

Propagation of neuropathic pain is mimicked by the induction of spinal nerve ligation (SNL) injury in the rat animal models using an amended procedure described by Kim and Chung, (Kim, et al., Pain 50:355-363 (1992), and generally illustrated as follows. Anesthesia is induced with 2% halothane in oxygen at 2 L/min and maintained with 0.5% halothane in oxygen. After surgical preparation of the rats and exposure of the dorsal vertebral column from L4 to S2, the L5 and L6 spinal nerves are tightly ligated at a position distal to the dorsal root ganglion using a 4-0 silk suture. The incision is then closed and the animals are allowed to recover for five days. Rats that exhibit motor deficiencies, such as paw-dragging, or rats that fail to exhibit subsequent tactile allodynia are excluded from further testing. Control rats undergo the same operation and handling as the experimental animals, but without SNL. Animal baseline withdrawals from tactile and thermal stimuli as described below are recorded. Tactile testing is performed, for example, using von Frey filaments and thermal testing is performed, for example, using an infrared heat source. Animals that undergo the L5/L6 spinal nerve ligation surgery are tested for tactile and thermal hypersensitivities on Day 3 through Day 7 after surgery. On Day 8, animals receive intrathecal administration of morphine, naltrexone or morphine and naltrexone at one of several ratios. Animals are tested, for example, 20 to 30 minutes after intrathecal administration (repeated daily, for example, for 7 days) for anti-allodynia and hyperalgesic effects. Three days after the last day of drug administration and testing, animals are retested for a return to post surgery baseline latencies/thresholds.

For an initial study, fifty male rats prepared as described above undergo the surgery for ligation of L5 and L6 spinal nerves. Five groups of 8 rats receive intrathecal injections twice daily for 7 days. The dose groups are as follows: 1) vehicle, 2) morphine (10 μg/dosing interval), 3) morphine (10 μg/dosing interval) plus naltrexone at 1:100,000 of morphine dose (0.1 ng), 4) morphine (10 μg/dosing interval) plus naltrexone at 1:1,000,000 of morphine dose (0.01 ng), and 5) naltrexone only at the same dose as group 3 (1:100,000) (0.1 ng).

The assessment of tactile allodynia, as indicated by a decreased threshold to paw-withdrawal following probing with non-noxious mechanical stimuli, is conducted by measuring the withdrawal threshold of the paw ipsilateral to the site of nerve injury in response to probing with a series of (e.g., 8) calibrated von Frey filaments (Stoetting, Wooddale, Ill.) in logarithmically spanned increments ranging from 0.41 to 15 g (4-150 n). While the rats are kept in suspended wire-mesh cages, each von Frey filament is applied perpendicularly to the plantar surface of the ligated paw. Measurements are taken, for example, both before and after administration of a drug or vehicle. Withdrawal threshold is determined by sequentially increasing and decreasing the stimulus strength (“up and down” method) and the threshold is analyzed using a Dixon non-parametric test as described by Chaplan and colleagues (Chaplan et al., J. Neurosci Methods 53:55-63 (1994). Measurements are expressed as the mean withdrawal threshold. Significant changes from baseline control values are detected by ANOVA followed by the post hoc least significance test. Significance is set at P≦0.05.

To assess paw-withdrawal latency to a thermal stimuli, an amended method described by Hargreaves and colleagues (Hargreaves et aL 1988) is employed, and generally illustrated as follows. Rats are allowed to acclimate within a plexiglass enclosure on a clear glass plate that is maintained at 30° C. for 30 minutes. A radiant heat source (e.g., infrared heat source), such as a high intensity projector lamp, is activated with a timer and focused onto the plantar surface of the hindpaw of a rat. Paw-withdrawal latency is determined by a photocell (e.g., a motion detector) that halts both the lamp and timer when the paw is withdrawn. The latency to withdrawal of the paw from the radiant (e.g., infrared) heat source is determined, for example, both before and after drug or vehicle administration. Baseline latencies are established, e.g, at about 16 seconds, to allow for detection of possible hyperalgesia. A maximal cut-off of 40 seconds is employed to prevent tissue damage. Significant changes from baseline control values are detected by ANOVA followed by the post hoc items least significance test. Significance is set at P≦0.05.

Results of the above-described initial study are as follows. Baseline latencies on day 7 after L5/L6 SNL demonstrated significant mechanical and thermal hypersensitivities. In this initial study, animals were divided into five groups and received either vehicle, morphine (10 μg), morphine (10 μg) and naltrexone (0.1 ng), morphine (10 μg) and naltrexone (0.01 ng), or naltrexone (0.1 ng) alone. The combination of morphine (10 μg) and naltrexone (0.1 ng) representing a ratio of 1:100,000 (naltrexone:morphine) resulted in a significant antihyperalgesic effect as compared to morphine (10 μg) alone on day 8 through day 14 when the area under the curves are compared. The combination of morphine (10 μg) and naltrexone (0.01 ng) representing a ratio of 1:1,000,000 (naltrexone:morphine) resulted in a significant antihyperalgesic effect as compared to vehicle or naltrexone alone but was not significantly different from morphine (10 μg) alone on day 8 through day 14. Although morphine alone at 10 μg resulted in 65 and 73% antihyperalgesia on day 8 and 9, respectively, with return to baseline by day 12, the combination of morphine (10 μg) and naltrexone (0.1 ng) (1:100,000) resulted in 71, 91, 90, 76, 86 and 69% antihyperalgesia on days 8 through 13, respectively, as well as analgesia (paw withdrawal latencies went above baseline) days 8, 9 and 10. Morphine alone resulted in tolerance to the antihyperalgesia effect by day 11 (38% activity), whereas the combination of morphine (10 μg) and naltrexone (0.1 ng) (1:100,000) resulted in an effect over 70% through day 13 and over 50% on day 14. Both vehicle and naltrexone alone had no effect on either antihyperalgesia or allodynia. More importantly, it is well know that morphine given alone by the intrathecal route has no antiallodynic effect (similar to what is reported clinically) yet the combination of morphine (10 μg) and naltrexone (0.1 ng) (1:100,000) resulted in a statistically significant effect on day 9 (p>0.05) with trends towards significance on days 8, 10 and 11. Additional experiments with various doses and times of testing are performed to further reveal the antiallodynia efficacy of the morphine and naltrexone combination in a model of neuropathic pain.

B. Additional SNL Study

Neuropathic surgery is performed as described above and neuropathic injury is produced by tightly ligating the left L5 and L6 spinal nerves under gaseous anesthesia with a mixture of halothane (3% for induction and 2% for maintenance) and O₂. Following recovery, development of neuropathic pain is evaluated daily (for 1 week or longer) by measuring mechanical sensitivity of the injured paw to von Frey filaments with incremental bending forces (0.1-15 g) as described by Chaplan et al., 1994 as described above. Animals are considered to be neuropathic when they show full-blown mechanical allodynia behavior (paw flinch behavior response to the lowest bending force applied). When animals show consistent mechanical allodynia behavior for 2 days, this is taken as baseline value. Test drug or vehicle is then administered spinally and mechanical threshold for paw flinching is measured again, for example, at 30, 60, 90 and 120 minutes after dosing, on the injured side. The measurement of antihyperalgesia is made as described above by the method of Hargreaves et al. (1988) to assess paw withdrawal latency to a thermal nociceptive stimulus. The measurement of antiallodynia is made as described above by behavioral testing of tactile hypersensitivity.

In this additional study, animals underwent intrathecal implantation of a catheter as described above that allows drug delivery at the lumbar (L5) spinal cord region. Animals allowed to recover for 5 days (with animals displaying motor trouble or paralysis removed from the study) are tested. Animal baseline withdrawals from thermal and tactile stimuli are recorded. Thermal testing is performed using an infrared heat source and tactile testing is performed using von Frey filaments as described above. Animals undergoing the L5/L6 spinal nerve ligation surgery are tested for tactile and thermal hypersensitivities on Day 5 and Day 7 after surgery. On Day 8 post-surgery, animals are divided into four groups and received intrathecal administration of vehicle morphine (10 μg) and naltrexone (0.1 ng) twice daily, morphine (10 μg) and naltrexone (0.33 ng) twice daily or morphine (10 μg) and naltrexone (0.33 ng) once daily. The first day of compound administration is noted as Day 1. Animals are tested 20-30 minutes after intrathecal administration for anti-allodynic and hyperalgesic effects, as described above, with intrathecal administration and subsequent testing daily for 7 days. Three days after the last day of drug injection and testing, animals are retested for a return to post-surgery baseline latencies/thresholds as described above.

Again, in this study, baseline latencies on Day 7 after L5/L6 SNL demonstrated significant mechanical and thermal hypersensitivities. Vehicle had no effect on hyperalgesia or allodynia. The vehicle group from this additional study and the vehicle-group from the initial study described above were not statistically different and were pooled for analysis. This allowed statistical comparison of the combination (e.g., morphine and naltrexone) dose groups with the naltrexone alone and morphine alone groups from the initial study.

The combination of morphine (10 μg) and naltrexone (0.1 ng) representing a ratio of 1:100,000 (naltrexone:morphine) twice daily resulted in a significant anti-hyperalgesic effect compared to vehicle or morphine (10 μg) alone for the Day 1 through Day 7 duration as assessed by the areas under the time curves. The combination of morphine (10 μg) and naltrexone (0.33 ng) representing a ratio of 1:33,000 (naltrexone:morphine) administered either once or twice daily also resulted in a significant anti-hyperalgesic effect (p<0.001) compared to vehicle and morphine (10 μg) alone by this analysis.

Although morphine alone at 10 μg resulted in 65 and 73% anti-hyperalgesia on Day 1 and 2, respectively, with return to baseline by day 5, the combination of morphine (10 μg) and naltrexone (0.1 ng) (1:100,000 twice daily) resulted in 75, 81, 91, 63, 79, 67 and 56% anti-hyperalgesia on Days 1 through 7, respectively, as well as analgesia (paw withdrawal latencies went above baseline) Days 1 through 7. Morphine alone resulted in tolerance to the anti-hyperalgesia effect by Day 4 (38% activity), whereas the combination of morphine (10 μg) and naltrexone (0.1 ng) (1:100,000) resulted in a decrease in hyperalgesia of over 70% through day 5 and over 50% on Days 6& 7. As stated above, morphine given alone by the intrathecal route has no antiallodynic effect (similar to what is reported in humans) yet morphine and naltrexone (1:100,000) resulted in a statistically significant effect on Day 1 (p=0.02) with trends (p=0.15) toward efficacy on Days 2 and 3.

The combination of morphine (10 μg) and naltrexone (0.33 ng) (representing a ratio of 1:33,000 (naltrexone:morphine) twice daily) resulted in 100, 94, 91, 100, 89, 87 and 88% anti-hyperalgesia on Days 1 through 7, respectively, as well as analgesia (paw withdrawal latencies were longer than baseline) on Days 1 through 7. No significant tolerance was seen with the combination of morphine (10 μg) and naltrexone (0.33 ng) (1:33,000 twice daily). Morphine (10 μg) and naltrexone (0.33 ng) (1:33,000 twice daily) resulted in a statistically significant anti-allodynic effect on Day 1 (p=0.02) with trends (p=0.06) toward efficacy on Days 2 and 3.

The combination of morphine (10 μg) and naltrexone (0.33 ng) (1:33,000 once daily) resulted in 96, 79, 92, 75, 79, 65 and 77% anti-hyperalgesia on Days 1 through 7, respectively, as well as analgesia (paw withdrawal latencies were higher than baseline, pre-neuropathic levels) on Days 1 through 7. No significant tolerance was seen from Day 1 through Day 5. The slightly decreased effect on Day 6 could reflect a decrease in potency, but the 77% anti-hyperalgesia observed the next day (Day 7) suggests a lack of tolerance overall for this treatment group. The combination of morphine (10 μg) and naltrexone (0.33 ng) (1:33,000 once daily) resulted in a statistically significant anti-allodynic effect on Day 1 (p=0.02) with trends (p=0.06) of being effective on Days 2 and 3.

Other well known rodent models for neuropathic pain which may be used include the chronic constriction injury by loose ligation of the sciatic nerve (CCI) model, as described by Bennet and Xie (Bennet and Xie, 1988); the tight ligation of the partial sciatic nerve (PSL) model, as described by Seltzer (Seltzer et al., 1990); and more recently, the partial denervation model, as described by Decosterd (Decosterd et al., 2000), wherein two of the three terminal distal branches of the sciatic nerves i.e., tibial and common peroneal nerves, are axotomized and one nerve i.e., the sural nerve, is spared. These animal models are effective in mimicking neuropathic pain for in vivo evaluation of the compounds or compositions presented herein.

### EXAMPLE 2

### Dynorphin Studies

From in vivo rat L5/L6 spinal nerve ligation (SNL) study described in Example 1, dynorphin is assayed and quantified as follows.

To determine the amount of dynorphin A produced within the in vivo animal models, an amended procedure described by Vanderah and colleagues (Vanderah et al., Journal of Neuroscience 20:7074-7079 (2000) is employed, and generally illustrated as follows. Rats are deeply anesthetized with ether and decapitated on day 7 of drug or vehicle administration. The spinal cord is injected with ice-cold saline and placed on an iced glass Petri dish, and the lumbar cord is rapidly dissected. These tissue samples are immediately frozen on dry ice and stored at −70° C. Thawed tissue is placed in IN acetic acid, homogenized with a Polytron, and then incubated for 20-30 minutes at 95° C. After centrifugation at 10,000-14,000xg for 20 minutes at 4° C., the supernatant is lyophilized and stored at −70° C. Protein concentrations are determined by the use of the bicinchoninic acid method with bovine serum albumin as a standard. The immunoassay is performed by the use of a commercial enzyme immunoassay kit (e.g., Peninsula Laboratories, Belmont, Calif.) with an antibody specific for dynorphin A. Standard curves are constructed and the dynorphin content is determined. Differences in dynorphin content between treatment groups are determined using accepted statistical methods. Pair-wise comparisons between drug or vehicle administration is detected by the Student's t-test. Significance is determined at thep <0.05 level.

### EXAMPLE 3

### G-Protein Signaling Studies

In the following example, an evaluation of the correlation or relationship between increased dynorphin release in neuropathic pain and G-protein excitatory signaling of opioid receptors is assessed. G-protein studies are performed using rats exhibiting neuropathic pain behavior following L5/L6 spinal nerve ligation as described in Example 1. Opioid agonist and antagonist combinations are tested for their ability to prevent putative G-protein excitatory signaling in neuropathic pain.

For the induction of morphine tolerance, male Sprague-Dawley rats, weighing approximately 200-250 grams, are administered morphine (10 mg/kg, SC) or saline twice daily separated by at least 5 hours for 7 days. Tissue from spinal cord is harvested and dissected 12 hours after the last injection. Membrane preparations from different spinal cord preparation from the same animals is stimulated with morphine or with a mixture of naltrexone: morphine in varying ratios (e.g., 1:50, 1:100, 1:1000 1:10,000, 1:100,000, 1:1,000,000, 1:10,000,000). In an initial experiment, the naltrexone: morphine ratio is 1:1,000,000.

Synaptic membranes are prepared from rat brains in well known procedures (Friedman et al., Anal. Biochem 214:171-178 (1995); Wang et al, J Pharmacol Exper Therap 273:492-498 (1995). Brain and/or other central nervous system (CNS) regions of interest are homogenized in 10 volumes of ice-cold homogenization solution containing 25 mM HEPES (pH 7.5), 1 mM EGTA, 100 mM sucrose, 50 μg/mL leupeptin, 10 μg/mL aprotinin, 2 μg/mL soybean trypsin inhibitor, 0.04 mM PMSF, and 0.2% 2-mercaptoethanol using a Teflon-glass homogenizer. Homogenates are centrifuged at 100 g at 4° C. for 10 min and the resulting supernatant is centrifuged at 50,000 g at 4° C. for 20 min. The obtained pellet is washed and suspended in 5 mL of a reaction solution containing 25 mM HEPES (pH 7.5), 1 mM EGTA, 100 mM NaCl, 1 mM MgCl₂, 50 μg/mL leupeptin, 10 μg/mL aprotinin, 2 μg/mL soybean trypsin inhibitor, 0.04 mM PMSF, and 0.2% 2-mercaptoethanol. Protein concentration within the membrane suspensions is determined by Bradford analysis.

The activation of μ-opioid receptor-associated G-proteins is assessed by the ability of μ-opioid receptor stimulation to increase [³⁵S]-GTPγS binding to the receptor associated Ga proteins in well established assays ((Friedman et al., Anal. Biochem 214:171-178 (1995); Wang et al, i J Pharmacol Exper Therap 273:492-498 (1995); Jin et al, J Neurochem 78:1-11 (2001). Synaptic membranes (200 μg) prepared from brain and/or other CNS regions of interest are incubated with increasing concentration of morphine or DAMGO for 5 min at 37° C. in the presence of 0.5 nM of [³⁵S]-GTPγS. Membranes are solubilized as described above and the [³⁵S]-GTPγS-bound Gα proteins are isolated by immunoprecipiation with specific anti-Gα antibodies followed by protein A/G-conjugated agarose. The affinity and efficacy of the agonist is calculated from concentration-effect relationships.

In another assay, the linkage between μ-opioid receptors and G-proteins is assessed, for example, by co-immunoprecipitation under both basal and receptor-stimulated conditions. Gα proteins that are coupled to μ-opioid receptors are immunoprecipitated with specific antibodies to the Gα antibodies and the receptor protein are detected using anti-μ-opioid receptor. Synaptic membranes (200 μg) prepared from brain and/or other CNS regions of interest are incubated with DAMGO for 5 min at 37° C. Membranes are solubilized in a reaction buffer containing 0.5% digitonin, 0.2% sodium cholate, and 0.5% NP-40; and G-protein coupled μ-opioid receptors are isolated by immunoprecipitation with various specific antibodies to Gαi, Gαo, Gαs, or Gαq using well-known methods (Gurdal, et al., Mol Pharmacol 47:772-778 (1995); Cai, et al., Mol Pharmacol 56:989-996 (1995); Jin et al, J Neurochem 78:1-11 (2001). Receptors that co-precipitate with selective Gα protein(s) are identified and quantified using commercially available specific antibodies directed against the μ-opioid receptor. Specifically, the receptor-G protein coupling profiles in the tested groups are determined by examining the Gα protein subclasses in μ-opioid receptor immunoprecipitates of lysates derived from difference brain regions. The specificity of the receptor antibody is determined prior to its use in the experiments described herein. In addition, the specificity of the receptor-G protein coupling profiles (under receptor-stimulation conditions) is tested for sensitivity to blockade by the selective μ-opioid receptor antagonist, β-funatrexamine.

Tests of agonist-induced association of Gβγ with adenylyl cyclase subtypes (e.g., II and/or IV) are performed. It is known that type II and IV adenylyl cyclases are activated by Gβγ that results from the dissociation of G proteins following receptor stimulation and different combinations of the identified multiple Gβ and Gγ subunits may be released, which may have differential efficacies for activating adenylyl cyclases. Evaluations of the adenylyl cyclase subtypes (e.g., II and/or IV), that couples to Gβγ following μ-opioid receptor stimulation are made. Synaptic membranes (200 μg) prepared from spinal cord dorsal horn are incubated with varying concentration of morphine and 1 nM Gpp(NH)p for 5 min at 37° C. (Friedman and Wang, 1996, supra). Membranes are solubilized and Gβγ is isolated by immunoprecipitation with specific anti-Gβ antibody and the level of adenylyl cyclase subtypes in the Gβ immunoprecipitate is assessed by Western analysis using specific antibodies directed against adenylyl cyclase subtypes (e.g., II and/or IV). Antibodies against other adenylyl cyclase subtypes are used as controls. Results are confirmed by a separate set of experiments in which the Gβ subunit(s) in the identified adenylyl cyclase immunoprecipitates is tested using Gβ subtype-specific antibodies. The specificity of the receptor stimulation is defined by testing for receptor sensitivity with specific opioid receptor antagonists including the selective μ-opioid receptor antagonist, β-funatrexamine.

While the invention will be described in connection with one or more embodiments, it will be understood that the invention is not limited to those embodiments. On the contrary, the invention includes all alternatives, modification, and equivalents as may be included within the spirit and scope of the appended claims.

1. A method for treating neuropathic pain in a patient in need thereof comprising administering to the patient a composition comprising an amount of an opioid antagonist effective to alleviate the neuropathic pain.

2. The method of claim 1 wherein the composition additionally comprises an opioid agonist and optionally a pharmaceutically acceptable carrier or excipient.

3. The method of claim 1 or 2 wherein the amount of the antagonist is less than an effective antagonistic amount.

4. The method of claim 2 wherein the excitatory opioid receptor antagonist or the agonist is present as a pharmaceutically acceptable salt.

5. The method of claim 1 or 2 wherein the antagonist is naloxone.

6. The method of claim 1 or 2 wherein the antagonist is naltrexone.

7. The method of claim 1 or 2 wherein the antagonist is nalmefene.

8. The method of claim 2 wherein the amount of the agonist is an analgesic or a subanalgesic amount.

9. The method of claim 2 wherein the agonist is morphine, hydrocodone, oxycodone, codeine, fentanyl, alfentanil, hydromorphone, meperidine, methadone, oxymorphone, propoxyphene, or tramadol.

10. The method of claim 2 wherein the agonist is morphine.

11. The method of claim 2 wherein the agonist is hydrocodone.

12. The method of claim 2 wherein the agonist is oxycodone.

13. The method of claim 2 wherein the agonist is tramadol.

14. The method of claim 2 wherein the antagonist is naltrexone and the agonist is morphine.

15. The method of claim 2 wherein the antagonist is naltrexone and the agonist is oxycodone.

16. The method of claim 2 wherein the antagonist is naltrexone and the agonist is hydrocodone.

17. The method of claim 2 wherein the antagonist is naltrexone and the agonist is tramadol.

18. The method of claim 2 wherein the antagonist is nalmefene and the agonist is morphine.

19. The method of claim 2 wherein the antagonist is nalmefene and the agonist is oxycodone.

20. The method of claim 2 wherein the antagonist is nalmefene and the agonist is hydrocodone.

21. The method of claim 2 wherein the antagonist is nalmefene and the agonist is tramadol.

22. The method of claim 1 or 2 wherein the composition further comprises a therapeutically effective amount of at least one anticonvulsant.

23. The method of claim 1 or 2 wherein the composition further comprises an anticonvulsant that is lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan, or L-5-hydroxytryptophan.

24. The method of claim 1 or 2 wherein the composition further comprises a therapeutically effective amount of at least one non-narcotic analgesic.

25. The method of claim 1 or 2 wherein the composition further comprises a therapeutically effective amount of non-steroidal anti-inflammatory drug.

26. The method of claim 1 or 2 wherein the composition further comprises a nonsteroidal anti-inflammatory drug that is aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxican, sulindac, tolmetin, or zomepirac.

27. The method of claim 1 or 2 wherein the composition further comprises tricyclic antidepressant that is amitriptyline, imipramine, desipramine or nortriptyline.

28. The method of claim 1 or 2 wherein the composition further comprises a therapeutically effect amount of at least one glutamate receptor antagonist.

29. The method of claim 1 or 2 wherein the composition further comprises a glutamate receptor antagonist that is that is ketamine, MK801, memantine, dextromethorphan, dextrorphan, LY293558, LY382884, amantadine, agmatine, aptiganel, gavestinel, selfotel, 7-chlorokynurenate, MRZ 2/579, MDL 105,519, riluzole, CPP, AP5, APV, NBQX, CNQX or trans-ACPD.

30. The method of claim 1 or 2 wherein the composition further comprises a therapeutically effective amount of at least one anti-dynorphin agent.

31. The method of claim 1 or 2 wherein the composition further comprises an anti-dynorphin agent that is anti-dynorphin antibodies, soluble kappa opioid receptors, or soluble kappa opioid receptor fusion proteins.

32. The method of claim 1 or 2 wherein the composition further comprises a therapeutically effective amount of at least one nicotinic receptor antagonist.

33. The method of claim 1 or 2 wherein the composition further comprises a therapeutic effective amount of at least one local anesthetic.

34. The method of claim 1 or 2 wherein the composition further comprises a local anesthetic that is bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocaine hydrochloride, procaine hydrochloride, propoxycaine hydrochloride tetracaine, or tetracaine hydrochloride.

35. The method of claim 1 or 2 wherein the composition further comprises at least one colloidal dispersion system.

36. The method of claim 1 or 2 wherein the composition further comprises at least one additive or preservative.

37. The method of claim 1 or 2 wherein the composition further comprises at least one pharmaceutically acceptable diluent.

38. The method of claim 1 or 2 wherein the composition further comprises at least one binder.

39. The method of claim 1 or 2 wherein the composition further comprises at least one plasticizer.

40. The method of claim 1 or 2 wherein the pharmaceutically acceptable carrier in the composition is a slow release agent.

41. The method of claim 1 or 2 wherein the composition is administered orally to the patient.

42. The method of claim 1 or 2 wherein the composition is administered intravenously to the patient.

43. The method of claim 1 or 2 wherein the composition is administered intrathecally or epidurally to the patient.

44. The method of claim 1 or 2 wherein the composition is administered intramuscularly to the patient.

45. The method of claim 1 or 2 wherein the composition is administered subcutaneously to the patient.

46. The method of claim 1 or 2 wherein the composition is administered perineurally to the patient.

47. The method of claim 1 or 2 wherein the composition is administered intradermally to the patient.

48. The method of claim 1 or 2 wherein the composition is administered topically or transcutaneously to the patient.

49. The method of claim 1 or 2 wherein the patient is a mammal.

50. The method of claim 1 or 2 wherein the patient is a human.

51. The method of claim 1 or 2 wherein the administration is from one time daily to four times daily.

52. The method of claim 1 or 2 wherein the administration is from two times daily to four times daily.

53. The method of claim 1 or 2 wherein the administration is from one time daily to two times daily.

54. The method of claim 1 or 2 wherein alleviation of the neuropathic pain is indicated by alleviation of allodynia.

55. The method of claim 1 or 2 wherein alleviation of the neuropathic pain is indicated by alleviation of hyperalgesia.

56. The method of claim 1 or 2 wherein alleviation of the neuropathic pain is indicated by alleviation of spontaneous burning pain.

57. The method of claim 1 or 2 wherein alleviation of the neuropathic pain is indicated by alleviation of phantom pain.

58. The method of claim 1 or 2 wherein alleviation of the neuropathic pain is indicated by alleviation of hyperesthesia.

59. The method of claim 1 or 2 wherein the neuropathic pain is associated with migraine.

60. The method of claim 1 or 2 wherein the neuropathic pain is associated with diabetes.

61. The method of claim 1 or 2 wherein the neuropathic pain is associated with diabetic neuropathy.

62. The method of claim 1 or 2 wherein the neuropathic pain is associated with shingles.

63. The method of claim 1 or 2 wherein the neuropathic pain is associated with burn injury.

64. The method of claim 1 or 2 wherein the neuropathic pain is associated with opthalmic injury.

65. The method of claim 1 or 2 wherein the neuropathic pain is associated with oral nerve injury or damage.

66. The method of claim 1 or 2 wherein the neuropathic pain is associated with oral nerve injury and wherein the oral nerve injury is caused by endodontic procedures.

67. The method of claim 1 or 2 wherein the neuropathic pain is associated with sensory nerve injury or damage.

68. The method of claim 1 or 2 wherein the neuropathic pain is associated with reflex sympathetic dystrophy (RSD).

69. The method claim 1 or 2 wherein the neuropathic pain is associated with post-herpetic neuralgia.

70. The method of claim 1 or 2 wherein the neuropathic pain is associated with arthritis.

71. The method of claim 1 or 2 wherein the neuropathic pain is associated with cancer.

72. The method of claim 1 or 2 wherein the neuropathic pain is not associated with the administration of a therapeutic agent.

73. A method for treating neuropathic pain in a patient in need thereof comprising administering to the patient a composition an amount of naltrexone, nalmefene or naloxone from about 0.000001 mg to less than about 1.0 mg and an amount of morphine, oxycodone, oxymorphone, hydrocodone or tramadol from about 0.1 mg to about 300 mg.

74. A method for treating neuropathic pain in a patient in need thereof comprising administering to the patient a composition an amount of naltrexone, nalmefene or naloxone from about 1 fg to less than about 1 ng and an amount of morphine, oxycodone, oxymorphone, hydrocodone or tramadol from about 0.1 mg to about 300 mg.

75. A method for treating hyperesthesia in a patient in need thereof comprising administering to the patient a composition comprising an amount of an opioid antagonist effective to alleviate the hyperesthesia.

76. A method for treating hyperalgesia in a patient in need thereof comprising administering to the patient a composition comprising an amount of an opioid antagonist effective to alleviate the hyperalgesia.

77. A method for treating allodynia in a patient in need thereof comprising administering to the patient a composition comprising an amount of an opioid antagonist effective to alleviate the allodynia.

78. A method for treating spontaneous burning pain in a patient in need thereof comprising administering to the patient a composition comprising an amount of an opioid antagonist effective to alleviate the spontaneous burning pain.

79. A method for treating phantom pain in a patient in need thereof comprising administering to the patient a composition comprising an amount of an opioid antagonist effective to alleviate the phantom pain.

80. A method for treating pain in a subject with neuropathic pain comprising administering to the subject an opioid agonist and an opioid antagonist, wherein the antagonist is administered in an amount effective to enhance the neuropathic pain-alleviating potency of the agonist.

81. The method of claim 80 wherein the potency of the agonist is measured by alleviation of hyperesthesia.

82. The method of claim 80 wherein the potency of the agonist is measured by alleviation of hyperalgesia.

83. The method of claim 80 wherein the potency of the agonist is measured by alleviation of allodynia.

84. The method of claim 80 wherein the potency of the agonist is measured by alleviation of spontaneous burning pain.

85. The method of claim 80 wherein the potency of the agonist is measured by alleviation of phantom pain.

86. The method of claim 80 wherein the amount of the agonist is an analgesic or subanalgesic amount.

87. The method of claim 80 wherein the agonist is morphine.

88. The method of claim 80 wherein the agonist is oxycodone.

89. The method of claim 80 wherein the agonist is hydrocodone.

90. The method of claim 80 wherein the agonist is oxymorphone.

91. The method of claim 80 wherein the agonist is hydromorphone.

92. The method of claim 80 wherein the agonist is tramadol.

93. The method of claim 80 wherein the antagonist is nalmefene.

94. The method of claim 80 wherein the antagonist is naltrexone.

95. The method of claim 80 wherein the antagonist is naloxone.

96. The method of claim 80 wherein the mode of administration is oral.

97. The method of claim 80 wherein the mode of administration is intravenous.

98. The method of claim 80 wherein the mode of administration is intrathecal or epidural.

99. The method of claim 80 wherein the mode of administration is intramuscular.

100. The method of claim 80 wherein the mode of administration is subcutaneous.

101. The method of claim 80 wherein the mode of administration is perineural.

102. The method of claim 80 wherein the mode of administration is intradermal.

103. The method of claim 80 wherein the mode of administration is topical.

104. The method of claim 80 wherein the mode of administration is transcutaneous.

105. The method of claim 80 wherein the agonist is oxycodone and the antagonist is naltrexone.

106. The method of claim 80 wherein the agonist is morphine and the antagonist is naltrexone.

107. The method of claim 80 wherein the agonist is oxycodone and the antagonist is nalmefene.

108. The method of claim 80 wherein the agonist is morphine and the antagonist is nalmefene.

109. The method of claim 80 wherein the agonist is oxycodone and the antagonist is naloxone.

110. The method of claim 80 wherein the agonist is morphine and the antagonist is naloxone.

111. The method of claim 80 wherein the amount of the agonist is from about 0.1 mg to about 300 mg.

112. The method of claim 80 wherein the amount of the antagonist is from about 0.000001 mg to about or less than about 1 mg.

113. The method of claim 80 wherein the amount of the antagonist is additionally effective to alleviate a tolerance, dependence, addiction or withdrawal effect of the agonist.

114. The method of claim 80 wherein the amount of the antagonist administered is at least 50 to 100 fold less than the amount of the agonist administered.

115. The method of claim 80 wherein the amount of the antagonist administered is at least 100 to 1000 fold less than the amount of the agonist administered.

116. The method of claim 80 wherein the amount of the antagonist administered is at least more than 40 fold less than the amount of the agonist administered.

117. The method of claim 80 wherein the amount of the antagonist administered is at least more than 50 fold less than the amount of the agonist administered.

118. The method of claim 80 wherein the amount of the antagonist administered is at least more than 100 fold less than the amount of the agonist administered.

119. The method of claim 80 wherein the amount of the antagonist administered is at least more than 1000 fold less than the amount of the agonist administered.

120. The method of claim 80 wherein the amount of the antagonist administered is at least more than 10,000 fold less than the amount of the agonist administered.

121. The method of claim 80 wherein the amount of the antagonist administered is at least more than 100,000 fold less than the amount of the agonist administered.

122. The method of claim 80 wherein the amount of the antagonist administered is at least more than 1,000,000 fold less than the amount of the agonist administered.

123. The method of claim 80 wherein the amount of the antagonist administered is at least more than 10,000,000 fold less than the amount of the agonist administered.

124. The method of claim 80 wherein the amount of the antagonist administered is at least more than 100,000,000 fold less than the amount of the agonist administered.

125. The method of claim 80 wherein the amount of the antagonist administered is at least more than 1,000,000,000 fold less than the amount of the agonist administered.

126. The method of claim 80 wherein the amount of the antagonist administered is at least more than 10,000,000,000 fold less than the amount of the agonist administered.

127. A method for enhancing the potency of an opioid agonist comprising administering to a subject with neuropathic pain an amount of the agonist and an amount of an opioid antagonist effective to enhance the neuropathic pain-alleviating potency of the agonist.

128. The method of claim 127 wherein the neuropathic pain-alleviating potency of the agonist by the antagonist is measured by alleviation of hyperesthesia.

129. The method of claim 127 wherein the neuropathic pain-alleviating potency of the agonist by the antagonist is measured by alleviation of hyperalgesia.

130. The method of claim 127 wherein the neuropathic pain-alleviating potency of the agtonist by the antagonist is measured by alleviation of allodynia.

131. The method of claim 127 wherein the neuropathic pain-alleviating potency of the agonist by the antagonist is measured by alleviation of spontaneous burning pain.

132. The method of claim 127 wherein the neuropathic pain-alleviating potency of the agonist by the antagonist is measured by alleviation of phantom pain.

133. The method of claim 127 wherein the amount of the agonist is an analgesic or subanalgesic amount.

134. The method of claim 127 wherein the agonist is morphine.

135. The method of claim 127 wherein the agonist is oxycodone.

136. The method of claim 127 wherein the agonist is hydrocodone.

137. The method of claim 127 wherein the agonist is oxymorphone.

138. The method of claim 127 wherein the agonist is hydromorphone.

139. The method of claim 127 wherein the agonist is tramadol.

140. The method of claim 127 wherein the antagonist is nalmefene.

141. The method of claim 127 wherein the antagonist is naltrexone.

142. The method of claim 127 wherein the antagonist is naloxone.

143. The method of claim 127 wherein the mode of administration is oral.

144. The method of claim 127 wherein the mode of administration is intravenous.

145. The method of claim 127 wherein the mode of administration is intrathecal or epidural.

146. The method of claim 127 wherein the mode of administration is intramuscular.

147. The method of claim 127 wherein the mode of administration is subcutaneous.

148. The method of claim 127 wherein the mode of administration is perineural.

149. The method of claim 127 wherein the mode of administration is intradermal.

150. The method of claim 127 wherein the mode of administration is topical.

151. The method of claim 127 wherein the mode of administration is transcutaneous.

152. The method of claim 127 wherein the agonist is oxycodone and the antagonist is naltrexone.

153. The method of claim 127 wherein the agonist is morphine and the antagonist is naltrexone.

154. The method of claim 127 wherein the agonist is oxycodone and the antagonist is nalmefene.

155. The method of claim 127 wherein the agonist is morphine and the antagonist is nalmefene.

156. The method of claim 127 wherein the agonist is oxycodone and the antagonist is naloxone.

157. The method of claim 127 wherein the agonist is morphine and the antagonist is naloxone.

158. The method of claim 127 wherein the amount of the agonist is from about 0.1 mg to about 300 mg.

159. The method of claim 127 wherein the amount of the antagonist is from about 0.000001 mg to about or less than about 1 mg.

160. The method of claim 127 wherein the amount of the antagonist is additionally effective to alleviate a tolerance, dependence, addiction or withdrawal effect of the agonist.

161. The method of claim 127 wherein the amount of the antagonist administered is at least 50 to 100 fold less than the amount of the agonist administered.

162. The method of claim 127 wherein the amount of the antagonist administered is at least 100 to 1000 fold less than the amount of the agonist administered.

163. The method of claim 127 wherein the amount of the antagonist administered is at least more than 40 fold less than the amount of the agonist administered.

164. The method of claim 127 wherein the amount of the antagonist administered is at least more than 50 fold less than the amount of the agonist administered.

165. The method of claim 127 wherein the amount of the antagonist administered is at least more than 100 fold less than the amount of the agonist administered.

166. The method of claim 127 wherein the amount of the antagonist administered is at least more than 1000 fold less than the amount of the agonist administered.

167. The method of claim 127 wherein the amount of the antagonist administered is at least more than 10,000 fold less than the amount of the agonist administered.

168. The method of claim 127 wherein the amount of the antagonist administered is at least more than 100,000 fold less than the amount of the agonist administered.

169. The method of claim 127 wherein the amount of the antagonist administered is at least more than 1,000,000 fold less than the amount of the agonist administered.

170. The method of claim 127 wherein the amount of the antagonist administered is at least more than 10,000,000 fold less than the amount of the agonist administered.

171. The method of claim 127 wherein the amount of the antagonist administered is at least more than 100,000,000 fold less than the amount of the agonist administered.

172. The method of claim 127 wherein the amount of the antagonist administered is at least more than 1,000,000,000 fold less than the amount of the agonist administered.

173. The method of claim 127 wherein the amount of the antagonist administered is at least more than 10,000,000,000 fold less than the amount of the agonist administered.

174. A composition for administration to a subject with neuropathic pain comprising an analgesic or subanalgesic amount of an opioid agonist and an amount of an opioid antagonist effective to enhance the neuropathic pain-alleviating potency of the agonist.

175. The composition of claim 174 wherein the neuropathic pain-alleviating potency of the agonist by the antagonist is measured by alleviation of hyperesthesia.

176. The composition of claim 174 wherein the neuropathic pain-alleviating potency of the agonist by the antagonist is measured by alleviation of hyperalgesia.

177. The composition of claim 174 wherein the neuropathic pain-alleviating potency of the agonist by the antagonist is measured by alleviation of allodynia.

178. The composition of claim 174 wherein the neuropathic pain-alleviating potency of the agonist by the antagonist is measured by alleviation of spontaneous burning pain.

179. The composition of claim 174 wherein the neuropathic pain-alleviating potency of the agonist by the antagonist is measured by alleviation of phantom pain.

180. The composition of claim 174 wherein the amount of the agonist is an analgesic or subanalgesic amount.

181. The composition of claim 174 wherein the agonist is morphine.

182. The composition of claim 174 wherein the agonist is oxycodone.

183. The composition of claim 174 wherein the antagonist is hydrocodone.

184. The composition of claim 174 wherein the agonist is oxymorphone.

185. The composition of claim 174 wherein the agonist is hydromorphone.

186. The composition of claim 174 wherein the agonist is tramadol.

187. The composition of claim 174 wherein the antagonist is nalmefene.

188. The composition of claim 174 wherein the antagonist is naltrexone.

189. The composition of claim 174 wherein the antagonist is naloxone.

190. The composition of claim 174 wherein the mode of a dministration is oral.

191. The composition of claim 174 wherein the mode of administration is intravenous.

192. The composition of claim 174 wherein the mode of administration is intrathecal or epidural.

193. The composition of claim 174 wherein the mode of administration is intramuscular.

194. The composition of claim 174 wherein the mode of administration is subcutaneous.

195. The composition of claim 174 wherein the mode of administration is perineural.

196. The composition of claim 174 wherein the mode of administration is intradermal.

197. The composition of claim 174 wherein the mode of administration is topical.

198. The composition of claim 174 wherein the mode of administration is transcutaneous.

199. The composition of claim 174 wherein the agonist is oxycodone and the antagonist is naltrexone.

200. The composition of claim 174 wherein the agonist is morphine and the antagonist is naltrexone.

201. The composition of claim 174 wherein the agonist is oxycodone and the antagonist is nalmefene.

202. The composition of claim 174 wherein the agonist is morphine and the antagonist is nalmefene.

203. The composition of claim 174 wherein the agonist is oxycodone and the antagonist is naloxone.

204. The composition of claim 174 wherein the agonist is morphine and the antagonist is naloxone.

205. A composition of claim 174 wherein the amount of the agonist is from about 0.1 mg to about 300 mg.

206. The composition of claim 174 wherein the amount of the antagonist is from about 0.000001 mg to about or less than about 1 mg.

207. The composition of claim 174 wherein the amount of the antagonist is additionally effective to attenuate the tolerance, dependence, addiction or withdrawal effects of the agonist.

208. The composition of claim 174 wherein the amount of the antagonist administered is at least 50 to 100 fold less than the amount of the agonist administered.

209. The composition of claim 174 wherein the amount of the antagonist administered is at least 100 to 1000 fold less than the amount of the agonist administered.

210. The composition of claim 174 wherein the amount of the antagonist administered is at least more than 40 fold less than the amount of the agonist administered.

211. The composition of claim 174 wherein the amount of the antagonist administered is at least more than 50 fold less than the amount of the agonist administered.

212. The composition of claim 174 wherein the amount of the antagonist administered is at least more than 100 fold less than the amount of the agonist administered.

213. The composition of claim 174 wherein the amount of the antagonist administered is at least more than 1000 fold less than the amount of the agonist administered.

214. The composition of claim 174 wherein the amount of the antagonist administered is at least more than 10,000 fold less than the amount of the agonist administered.

215. The composition of claim 174 wherein the amount of the antagonist administered is at least more than 100,000 fold less than the amount of the agonist administered.

216. The composition of claim 174 wherein the amount of the antagonist administered is at least more than 1,000,000 fold less than the amount of the agonist administered.

217. The composition of claim 174 wherein the amount of the antagonist administered is at least more than 10,000,000 fold less than the amount of the agonist administered.

218. The composition of claim 174 wherein the amount of the antagonist administered is at least more than 100,000,000 fold less than the amount of the agonist administered.

219. The composition of claim 174 wherein the amount of the antagonist administered is at least more than 1,000,000,000 fold less than the amount of the agonist administered.

220. The composition of claim 174 wherein the amount of the antagonist administered is at least more than 10,000,000,000 fold less than the amount of the agonist administered.

221. A composition for administration to a neuropathic pain patient comprising an amount of an opioid antagonist effective to enhance the neuropathic pain-alleviating potency of an endogenous opioid agonist.

222. The composition of claim 221 additionally comprising an opioid agonist and optionally a pharmaceutically acceptable carrier or excipient.

223. The composition of claim 221 wherein the amount of the antagonist is less than an effective antagonistic amount.

224. The composition of claim 221 or 222 wherein the antagonist or the agonist is present as a pharmaceutically acceptable salt.

225. The composition of claim 221 or 222 wherein the antagonist is naloxone.

226. The composition of claim 221 or 222 wherein the antagonist is naltrexone.

227. The composition of claim 221 or 222 wherein the antagonist is nalmefene.

228. The composition of claim 222 wherein the amount of the agonist is an analgesic or a subanalgesic amount.

229. The composition of claim 222 wherein the agonist is morphine, hydrocodone, oxycodone, codeine, fentanyl, alfentanil, hydromorphone, meperidine, methadone, oxymorphone, propoxyphene, or tramadol.

230. The composition of claim 222 wherein the agonist is morphine.

231. The composition of claim 222 wherein the agonist is hydrocodone.

232. The composition of claim 222 wherein the agonist is oxycodone.

233. The composition of claim 222 wherein the agonist is tramadol.

234. The composition of claim 222 wherein the antagonist is naltrexone and the agonist is morphine.

235. The composition of claim 222 wherein the antagonist is naltrexone and the agonist is oxycodone.

236. The composition of claim 222 wherein the antagonist is naltrexone and the agonist is hydrocodone.

237. The composition of claim 222 wherein the antagonist is naltrexone and the agonist is tramadol.

238. The composition of claim 222 wherein the antagonist is nalmefene and the agonist is morphine.

239. The composition of claim 222 wherein the antagonist is nalmefene and the agonist is oxycodone.

240. The composition of claim 222 wherein the antagonist is nalmefene and the agonist is hydrocodone.

241. The composition of claim 222 wherein the antagonist is nalmefene and the agonist is tramadol.

242. The composition of claim 221 or 222 further comprising a therapeutically effective amount of at least one anticonvulsant.

243. The composition of claim 221 or 222 further comprising an anticonvulsant that is lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan, or L-5-hydroxytryptophan.

244. The composition of claim 221 or 222 further comprising a therapeutically effective amount of at least one non-narcotic analgesic.

245. The composition of claim 221 or 222 further comprising a therapeutically effective amount of a non-narcotic analgesic that is a nonsteroidal anti-inflammatory drug.

246. The composition of claim 221 or 222 further comprising a nonsteroidal anti-inflammatory drug that is aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxican, sulindac, tolmetin or zomepirac.

247. The composition of claim 221 or 222 further comprising a tricyclic antidepressant that is amitriptyline, imipramine, desipramine or nortriptyline.

248. The composition of claim 221 or 222 further comprising a therapeutically effect amount of at least one glutamate receptor antagonist.

249. The composition of claim 221 or 222 further comprising a glutamate receptor antagonist that is ketamine, MK801, memantine, dextromethorphan, dextrorphan, LY293558, LY382884, amantadine, agmatine, aptiganel, gavestinel, selfotel, 7-chlorokynurenate, MRZ 2/579, MDL 105,519, riluzole, CPP, AP5, APV, NBQX, CNQX or trans-ACPD.

250. The composition of claim 221 or 222 further comprising a therapeutically effective amount of at least one anti-dynorphin agent.

251. The composition of claim 221 or 222 further comprising an anti-dynorphin agent that is anti-dynorphin antibodies, soluble kappa opioid receptors, or soluble kappa opioid receptor fusion proteins.

252. The composition of claim 221 or 222 further comprising a therapeutic effective amount of at least one local anesthetic.

253. The method of claim 221 or 222 wherein the composition further comprises a therapeutically effective amount of at least one nicotinic receptor antagonist.

254. The composition of claim 221 or 222 further comprising a local anesthetic that is bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocaine hydrochloride, procaine hydrochloride, propoxycaine hydrochloride tetracaine, or tetracaine hydrochloride.

255. The composition of claim 221 or 222 further comprising at least one colloidal dispersion system.

256. The composition of claim 221 or 222 further comprising at least one additive or preservative.

257. The composition of claim 221 or 222 further comprising at least one pharmaceutically acceptable diluent.

258. The composition of claim 221 or 222 further comprising at least one binder.

259. The composition of claim 221 or 222 further comprising at least one plasticizer.

260. The composition of claim 222 wherein the pharmaceutically acceptable carrier is a controlled release or sustained release agent.

261. The composition of claim 221 or 222 wherein the composition is in the form of oral formulation.

262. The composition of claim 221 or 222 wherein the composition is in the form of intravenous formulation.

263. The composition of claim 221 or 222 wherein the composition is in the form of a intrathecal or epidural formulation.

264. The composition of claim 221 or 222 wherein the composition is in the form of intramuscular formulation.

265. The composition of claim 221 or 222 wherein the composition is in the form of subcutaneous formulation.

266. The composition of claim 221 or 222 wherein the composition is in the form of perineural formulation.

267. The composition of claim 221 or 222 wherein the composition is in the form of intradermal formulation.

268. The composition of claim 221 or 222, wherein the composition is in the form of a topical formulation.

269. The composition of claim 221 or 222 wherein the composition is in the form of a capsule or tablet.

270. The composition of claim 221 or 222 wherein the patient is a mammal.

271. The composition of claim 221 or 222 wherein the patient is a human.

272. A composition for administration to a neuropathic pain patient comprising an amount of naltrexone, nalmefene or naloxone from about 0.000001 mg to less than about 1.0 mg and an amount of morphine, oxycodone, oxymorphone, hydrocodone or tramadol from about 0.1 mg to about 300 mg.
